(19) United States
(12) Patent Application Publication
(10) Pub. No.: US 2019/0000993 A1
(43) Pub. Date: Jan. 3, 2019

(54) TREATMENT OF DEVELOPMENTAL SYNDROMES

(71) Applicant: Ovid Therapeutics Inc., New York, NY (US)

(72) Inventor: Matthew DURING, Weston, CT (US)

(21) Appl. No.: 16/022,154

(22) Filed: Jun. 28, 2018

Related U.S. Application Data
(60) Provisional application No. 62/525,830, filed on Jun. 28, 2017.

(51) Int. Cl. A61K 48/00; C12N 15/62; C12N 15/113; A61K 31/7105; A61P 43/00

(52) U.S. Cl. CPC A61K 48/0016; C12N 15/62; A61P 43/00; A61K 31/7105; A61K 48/0075; C12N 15/113

(57) ABSTRACT

A method of treating a developmental syndrome in a patient in need thereof includes applying ultrasound to a target location in the patient's brain to enhance permeability of the patient's blood brain barrier at the target location and administering to the patient a vector encoding BDNF for delivery of BDNF to the target location, wherein the method provides improvement in at least one symptom of the developmental syndrome. Also provided is a method of treating a developmental syndrome in a patient in need thereof that includes administering to the patient an effective amount of a vector including (i) a constitutive promoter operatively linked to nucleic acid encoding BDNF, and (ii) a regulatory sequence including an AGRP promoter operatively linked to an interference RNA sequence, wherein the regulatory sequence down regulates expression of BDNF in response to BDNF induced physiological changes, and the method provides improvement in at least one symptom of the developmental syndrome.

### BACKGROUND

### 1. Technical Field

Treatment of developmental syndromes with gene therapy involving brain-derived neurotrophic factor.

### 2. Description of Related Art

Developmental syndromes range in severity and include disorders such as Prader-Willi syndrome, 16p11.2 deletion syndrome, 16p11.2 recurrent microdeletion, Albright hereditary osteodystrophy, Alström Syndrome, Bardet-Biedl syndrome, Borjeson-Forssman-Lehmann syndrome, Cohen syndrome, fragile X syndrome, fragile X syndrome (Prader-Willi Subtype), FMR1-Related Disorders, Down syndrome, Klinefelter syndrome, Turner syndrome, Smith-Magenis syndrome, Angelman syndrome, 21-Hydroxylase-Deficient Congenital Adrenal Hyperplasia, 2q37 Microdeletion syndrome, 3q29 Recurrent Deletion aka 3q29 Microdeletion syndrome, Achondroplasia, ADNP Syndrome aka ADNP-Related Intellectual Disability and Autism Spectrum Disorder, melanocortin 4 receptor (MC4R) disorder, Proopiomelanocortin Deficiency, 15q24 Microdeletion Syndrome, 15q Duplication Syndrome and Related Disorders, 1p36 Deletion Syndrome, and KANSL1-Related Intellectual Disability Syndrome aka Koolen de Vries syndrome.

Prader-Willi syndrome (PWS) is a genetic disease caused by lack of expression of genes from an imprinted region of the paternally inherited chromosome 15q11-q13, near the centromere (Aycan and Bas, J Clin Res Pediatr Endocrinol, 6(2):62-67 (2014)). The frequency of the disease is between about 1/10,000 and 1/30,000 with approximately 400,000 PWS patients living worldwide. PWS is a spectrum disorder which affects many systems in the body. Subjects with PWS typically suffer from a host of symptoms including neurologic, cognitive, endocrine, and behavioral abnormalities. Initially, infants exhibit hypotonia (floppy baby syndrome) and experience difficulty in sucking and feeding which can lead to growth delay. Subjects with PWS frequently have poor muscle tone, growth hormone deficiency, low levels of sex hormones, a constant feeling of hunger and excessive appetite (hyperphagia). They overeat, leading to weight gain, obesity and a high incidence of diabetes. Other signs appear including short stature, poor motor skills, underdeveloped sex organs, and mild intellectual and learning disabilities. PWS subjects may experience delayed speech and language development, and infertility. Behavioral symptoms may include cognitive impairment, cognitive rigidity, emotional lability and obsessive-compulsive behavior, with autistic symptomology, psychotic episodes, and biopolar disorder with psychosis. Additional clinical manifestations may include excessive daytime sleepiness, scoliosis, osteopenia/osteoporosis, decreased gastrointestinal motility, sleep disturbances, and reduced pain sensitivity.

Melanocortin 4 receptor (MC4R) deficiency (Melanocortin 4 receptor (MC4R) disorder) is the most common monogenic form of obesity. Farooqi et al., N Engl J Med. (2003) 348(12):1085-1095. Mutations in MC4R result in a distinct obesity syndrome that is inherited in a codominant manner. Id. Mutation carriers exhibit severe obesity, increased lean mass, increased linear growth, hyperphagia, and severe hyperinsulinemia. Id.

16p11.2 deletion syndrome is a disorder caused by a deletion of a small piece of chromosome 16. The deletion occurs near the middle of the chromosome at a location designated p11.2. Subjects with 16p11.2 deletion syndrome usually have developmental delay and intellectual disability. Most also have at least some features of autism spectrum disorders. These disorders are characterized by impaired communication and socialization skills, as well as delayed development of speech and language. Some subjects with this disorder have recurrent seizures (epilepsy). Some affected individuals have minor physical abnormalities such as low-set ears or partially webbed toes (partial syndactyly). People with this disorder are also at increased risk of obesity compared with the general population. However, there is no particular pattern of physical abnormalities that characterizes 16p11.2 deletion syndrome.

Albright's hereditary osteodystrophy is a syndrome with a wide range of manifestations including short stature, obesity, round face, subcutaneous (under the skin) ossifications (gradual replacement of cartilage by bone), and characteristic shortening and widening of the bones in the hands and feet (brachydactyly). The features of Albright's hereditary osteodystrophy are associated with resistance to parathyroid hormone (pseudohypoparathyroidism) and to other hormones (thyroid-stimulation hormone, in particular). This autosomal dominantly inherited condition is caused by mutations in the GNAS gene.

Alström syndrome is a rare genetic disorder that affects many body systems. Symptoms develop gradually, beginning in infancy, and can be variable. In childhood, the disorder is generally characterized by vision and hearing abnormalities, childhood obesity, and heart disease (cardiomyopathy). Over time, diabetes mellitus, liver problems, and slowly progressive kidney dysfunction which can lead to kidney failure may develop. Alström syndrome is caused by mutations in the ALMS1 gene.

Bardet-Biedl syndrome is an inherited condition that affects many parts of the body. People with this condition have progressive visual impairment due to cone-rod dystrophy; extra fingers or toes (polydactyly); truncal obesity; decreased function of the male gonads (hypogonadism); kidney abnormalities; and learning difficulties. Mutations in at least 14 genes are known to cause Bardet-Biedl syndrome.

Borjeson-Forssman-Lehmann syndrome (BFLS) is a genetic condition characterized by intellectual disability, obesity, seizures, hypogonadism, developmental delay and distinctive facial features. These symptoms are variable, even among members of the same family. BFLS is caused by mutations in the PHF6 gene on the X chromosome.

Cohen syndrome is a congenital condition whose main features are obesity, hypotonia (low muscle tone), intellectual disabilities, distinctive facial features with prominent upper central teeth and abnormalities of the hands and feet. However, the signs and symptoms present in people with Cohen syndrome may vary considerably. Although the exact cause of Cohen syndrome is unknown, some people with the condition have been found to have mutations in a gene called COH1 (also referred to as VPS13B).

Fragile X syndrome is characterized by developmental problems including intellectual disability and delayed speech and language development. Males are usually more severely affected than females. Additional features may include anxiety; attention deficit disorder (ADD); features of autism spectrum disorders that affect communication and social interaction; and seizures. Most males and some females with fragile X syndrome have characteristic physical features that become more apparent with age. These features may include a long and narrow face; large ears; a prominent jaw and forehead; unusually flexible fingers; flat feet; and in males, enlarged testicles (macroorchidism) after puberty. Mutations (changes) in the FMR1 gene cause fragile X syndrome (FXS). This gene carries instructions to make a protein called the fragile X mental retardation 1 protein. The FMR1 gene contains a CGG triplet repeat section of DNA, which normally repeats from 5 to around 40 times. In most cases of FXS, this section of DNA is repeated more than 200 times, which “turns off” the FMR1 gene and disrupts the function of the nervous system. In some cases, other types of changes in the FMR1 gene cause FXS. These changes may involve a deletion of all or part of the gene, or a change in the building blocks (amino acids) used to make the gene's protein. People with 55 to 200 repeats of the CGG segment are said to have an FMR1 premutation. Most people with a premutation are intellectually normal. In some cases, people with a premutation have lower levels of the gene's protein and may have some mild symptoms of FXS. About 20% of women with a premutation have premature ovarian failure, and some people with a premutation have an increased risk of developing fragile X-associated tremor/ataxia syndrome (FXTAS).

Down syndrome is a chromosome disorder associated with intellectual disability, a characteristic facial appearance, and low muscle tone in infancy. The degree of intellectual disability varies from mild to moderate. People with Down syndrome may also be born with various health concerns such as heart defects or digestive abnormalities. They also have an increased risk to develop gastroesophageal reflux, celiac disease, hypothyroidism, hearing and vision problems, leukemia, and Alzheimer disease. Down syndrome is caused by having three copies of chromosome 21.

Klinefelter syndrome (KS) is a condition that occurs in males when they have an extra X chromosome. Some males with KS have no obvious signs or symptoms while others may have varying degrees of cognitive, social, behavioral, and learning difficulties. Adults with Klinefelter syndrome may also experience primary hypogonadism (decreased testosterone production), small testes, enlarged breast tissue (gynecomastia), tall stature, and/or infertility. KS is not inherited, but usually occurs as a random event during the formation of reproductive cells (eggs and sperm).

Turner syndrome is a chromosomal disorder that affects development in females. It results when a female's cells has one normal X chromosome and the other X chromosome is either missing or structurally altered (females without Turner syndrome have two normal X chromosomes in each cell). Signs and symptoms may include short stature; premature ovarian failure; a “webbed” neck; a low hairline at the back of the neck; and swelling (lymphedema) of the hands and feet. Some people with Turner syndrome have skeletal abnormalities, kidney problems, and/or a congenital heart defect. Most affected girls and women have normal intelligence, but some have developmental delays, learning disabilities, and/or behavior problems.

Smith-Magenis syndrome is a developmental disorder that affects many parts of the body. The major features of this condition include mild to moderate intellectual disability, delayed speech and language skills, distinctive facial features, sleep disturbances, and behavioral problems. Most people with Smith-Magenis syndrome have a deletion of genetic material from a specific region of chromosome 17. Although this region contains multiple genes, it is believed that the loss of one particular gene, RAI1, in each cell is responsible for most of the characteristic features of the condition. Smith-Magenis syndrome is not typically inherited, but results from a genetic change that occurs during the formation of reproductive cells (eggs or sperm) or in early fetal development

Angelman syndrome is a genetic disorder that primarily affects the nervous system. Characteristic features of this condition include developmental delay, intellectual disability, severe speech impairment, problems with movement and balance (ataxia), epilepsy, and a small head size. Individuals with Angelman syndrome typically have a happy, excitable demeanor with frequent smiling, laughter, and hand-flapping movements. Many of the characteristic features of Angelman syndrome result from the loss of function of a gene called UBE3A.

21-Hydroxylase-Deficient Congenital Adrenal Hyperplasia is a genetic disorder of cortisol biosynthesis. It is caused by mutations in the human 21-hydroxylase gene (CYP21A2). Symptoms of 21-hydroxylase deficiency vary, but can involve salt-wasting crises in infants; ambiguous genitalia in female infants; excessive hair, deep voice, abnormal periods, no periods, and fertility problems in older girls and women; early development of masculine features in boys; and shorter than average adult height, acne, and blood pressure problems.

2q37 deletion or microdeletion syndrome is a chromosome disease that can affect many parts of the body. This condition is characterized by short stature, weak muscle tone (hypotonia) in infancy, mild to severe intellectual disability and developmental delay, autistic behavior, obesity, characteristic facial features, and other physical abnormalities, such as short bones of the hand and of 3-5 fingers, and abnormal lateral curvature of the spine (scoliosis). Other findings include seizures (20%-35%), congenital heart disease, brain abnormalities (hydrocephalus, dilated ventricles), umbilical/inguinal hernia, tracheomalacia, gastrointestinal abnormalities, and kidney malformations. 2q37 deletion syndrome is caused by a deletion of the genetic material from a specific region in the long (q) arm of chromosome 2. Most cases are not inherited.

3q29 microdeletion syndrome is caused by the loss of a small piece of DNA in one copy of chromosome 3. Symptoms may include delay reaching some developmental milestones such as sitting, walking or talking, frequent ear and respiratory infections, and a small head size (microcephaly). Some babies with this condition are born with a cleft lip or cleft palate, and a few have been reported to have heart defects. As children with this condition get older, they may develop behavioral difficulties such as autism, and they may have symptoms of mental illness. The severity of symptoms can vary, and some people with 3q29 microdeletion syndrome may have very mild symptoms or may not even know they are affected.

Achondroplasia is a disorder of bone growth that prevents the changing of cartilage (particularly in the long bones of the arms and legs) to bone. It is characterized by dwarfism, limited range of motion at the elbows, large head size, small fingers, and normal intelligence. Achondroplasia can cause health complications such as apnea, obesity, recurrent ear infections, and lordosis of the spine. Achondroplasia is caused by mutations in the FGFR3 gene.

ADNP syndrome, also known as Helsmoortel-van der Aa syndrome, is a complex neuro-developmental disorder that affects the brain and many other areas and functions of the body. ADNP syndrome can affect muscle tone, feeding, growth, hearing, vision, sleep, fine and gross motor skills, as well as the immune system, heart, endocrine system, and gastrointestinal tract. ADNP syndrome causes behavior disorders such as Autism Spectrum Disorder (ASD). ADNP is caused by a non-inherited (de novo) ADNP gene mutation. ADNP syndrome is thought to be one of the most common causes of non-inherited genetic autism

Proopiomelanocortin (POMC) deficiency is characterized by severe obesity that begins at an early age. Affected infants are usually a normal weight at birth, but they are constantly hungry, which leads to excessive feeding and weight gain during the first year and throughout life. In addition, people with this condition have low levels of adrenocorticotropic hormone (ACTH) which leads to adrenal insufficiency. They also tend to have red hair and pale skin. POMC deficiency is caused by mutations in the POMC gene. The condition is inherited.

Chromosome 2q24 microdeletion syndrome is a chromosome abnormality that occurs when there is a missing copy of the genetic material located on the long arm (q) of chromosome 2. The severity of the condition and the signs and symptoms depend on the size and location of the deletion and which genes are involved. Features that often occur in people with chromosome 2q deletion include developmental delay, intellectual disability, behavioral problems, and distinctive facial features, abnormality of the iris, bullet-shaped distal phalanx of the hallux, camptodactyly of finger, cleft palate, downslanted palpebral fissures, growth delay, hand clenching and intellectual disability.

15q24 Microdeletion Syndrome is a chromosome abnormality that occurs when there is a missing copy of the genetic material located on the long arm (q) of chromosome 15. The severity of the condition and the signs and symptoms depend on the size and location of the deletion and which genes are involved. Features that often occur in people with chromosome 15q deletion include developmental delay, intellectual disability, behavioral problems, and distinctive facial features. Most cases are not inherited, but people can pass the deletion on to their children.

15q duplication syndrome is a chromosome abnormality that occurs when an extra (duplicate) copy of the genetic material located on the long arm (q) of chromosome 15 is present in each cell. The severity of the condition and the associated signs and symptoms vary based on the size and location of the duplication and which genes are involved. Common features shared by many people with this duplication include developmental delay; intellectual disability; hypotonia (low muscle tone); seizures; high and/or cleft palate (roof of the mouth); scoliosis; slow growth; communication difficulties; behavioral problems; and distinctive facial features. Most cases are not inherited, although affected people can pass the duplication on to their children.

1p36 deletion syndrome is a chromosome disorder that typically causes severe intellectual disability. Most affected individuals do not speak, or speak only a few words. They may have temper tantrums, bite themselves, or exhibit other behavior problems. Most have structural abnormalities of the brain, and seizures occur in more than half of individuals with this disorder. Affected individuals usually have weak muscle tone (hypotonia) and swallowing difficulties (dysphagia). Other features include a small head that is unusually short and wide; vision and hearing problems; abnormalities of the skeleton, heart, gastrointestinal system, kidneys, or genitalia; and distinctive facial features. 1p36 deletion syndrome is caused by a deletion of genetic material from a specific region in the short (p) arm of chromosome 1. Most cases are not inherited; only about 20% of the cases of people with 1p36 deletion syndrome inherit the chromosome with a deleted segment from an unaffected parent.

KANSL1-Related Intellectual Disability Syndrome (Koolen de Vries syndrome) is a disorder characterized by developmental delay, mild to moderate intellectual disability, congenital malformations, and behavioral features. Developmental delay is noted from an early age. Other problems include weak muscle tone (hypotonia) in childhood, recurrent seizures (epilepsy), and distinctive facial features. Males with Koolen de Vries syndrome often have undescended testes (cryptorchidism). Other symptoms may include defects in the walls between the chambers of the heart (septal defects) or other heart defects, kidney problems, and skeletal anomalies such as foot deformities. It is caused by mutations in the KANSL1 gene, or by the loss of a small amount of genetic material in chromosome 17 that includes the KANSL1 gene (chromosome 17 q21.31 microdeletion).

Gene therapy involving delivery and regulation of brain-derived neurotrophic factor (BDNF) in the hypothalamus has been described in connection with the treatment of metabolic related disorders such as diabetes. See, e.g., U.S. Pat. No. 9,265,843, herein incorporated by reference in its entirety. See also, Cao, et al. Nat Med. 2009 April; 15(4):447-454, herein incorporated by reference in its entirety.

The blood brain barrier (BBB) prevents many compounds in the blood stream from entering the tissues and fluids of the brain. The BBB is formed by brain-specific endothelial cells and supported by the cells of the neurovascular unit to limit the passage of polar molecules or large molecules such as proteins and peptides into or out of the brain interstitium. However, the BBB also prevents many therapeutic compounds from entering the brain which can interfere with effective treatment of brain conditions and diseases. The BBB may interfere with delivery of therapeutic agents to the brain.

One method of assisting transport of therapeutic drugs through the BBB involves delivering ultrasound energy to the BBB which “opens up” the BBB and interferes with the ability of the BBB to prevent transport of therapeutic agents into the brain. See, e.g., U.S. Pat. No. 5,752,515, which is directed to image guided ultrasound delivery of compounds through the BBB. In one aspect, the change induced in the central nervous system (CNS) tissues and/or fluids by ultrasound is by heating or cavitation. Such heating or cavitation may present a drawback since it may cause damage to tissues and potentially degrade the compounds being delivered for therapeutic benefit. Ultrasound also causes degradation of organic compounds. See, e.g., Bremner et al., Current Organic Chemistry, 15(2): 168-177 (2011) (“Bremner et al.”). According to Bremner et al., when aqueous solutions are irradiated with ultrasound, the H—O bond in water is homolytically cleaved to form hydroxyl radicals and hydrogen atoms. This process is the result of cavitation, whereby very high temperatures and pressures are generated within an imploding bubble. Id. Accordingly, use of ultrasound in an attempt to open the BBB to cause or increase delivery of therapeutic compounds to the brain such as BDNF discussed above could degrade them and interfere with or prevent therapeutic treatment.

There remains a need for methods for treating developmental syndromes.

### SUMMARY

Methods of treating developmental syndromes with brain-derived neurotrophic factor (BNDF) are provided. In embodiments, a method of treating a developmental syndrome in a patient in need thereof includes applying ultrasound to a target location in the patient's brain to enhance permeability of the patient's blood brain barrier at the target location, and administering to the patient a vector encoding BDNF for delivery of BDNF to the target location, wherein the method provides improvement in at least one symptom of the developmental syndrome. In embodiments, a method of treating a developmental syndrome in a patient in need thereof includes administering to the patient an effective amount of a vector including (i) a constitutive promoter operatively linked to nucleic acid encoding BDNF, and (ii) a regulatory sequence including an AGRP promoter operatively linked to an interference RNA sequence, wherein the regulatory sequence down regulates expression of BDNF in response to BDNF induced physiological changes, and the method provides improvement in at least one symptom of the developmental syndrome.

In embodiments, the developmental syndrome is selected from the group consisting of Prader-Willi syndrome, 16p11.2 deletion syndrome, 16p11.2 recurrent microdeletion, Albright hereditary osteodystrophy, Alström Syndrome, Bardet-Biedl syndrome, Borjeson-Forssman-Lehmann syndrome, Cohen syndrome, fragile X syndrome, fragile X syndrome (Prader-Willi Subtype), FMR1-Related Disorders, Down syndrome, Klinefelter syndrome, Turner syndrome, Smith-Magenis syndrome, Angelman syndrome, 21-Hydroxylase-Deficient Congenital Adrenal Hyperplasia, 2q37 Microdeletion syndrome, 3q29 Microdeletion syndrome, Achondroplasia, ADNP Syndrome, melanocortin 4 receptor (MC4R) disorder, Melanocortin 4 receptor (MC4R) deficiency, Proopiomelanocortin Deficiency, 15q24 Microdeletion Syndrome, 15q Duplication Syndrome and Related Disorders, 1p36 Deletion Syndrome, and KANSL1-Related Intellectual Disability Syndrome. In embodiments, the developmental syndrome is melanocortin 4 receptor (MC4R) deficiency.

### BRIEF DESCRIPTION OF THE DRAWINGS

FIG. 1 is a schematic depiction of three rAAV vectors. First and uppermost is a depiction of a vector including a constitutive chicken ß-actin (CBA) promoter operatively linked to a nucleic acid sequence encoding yellow fluorescent protein, woodchuck post translational regulatory element (WPRE), and bovine growth poly-A, which can be flanked by AAV inverted terminal repeats. The next two schematics depict autoregulatory vectors containing two expression cassettes, one to express BDNF under a constitutive promoter, the other to express a microRNA targeting the same transgene driven by a promoter (AGRP484) responsive to BDNF-induced physiological changes. The second schematic includes a scrambled micro RNA (miRNA-scr) targeting no known genes. The third schematic includes a miRNA targeting BDNF (miRNA-Bdnf).

FIG. 2 depicts the nucleotide sequence of pAM/CBA-NPY-WPRE-BGH (SEQ ID NO: 1)

FIG. 3 depicts the nucleotide sequence of CAG-BDNF-HA-WPRE (SEQ ID NO:2)

FIG. 4 is a schematic depiction of a autoregulatory plasmid including a constitutive chicken ß-actin (CBA) promoter and a loxP sequence operatively linked to nucleic acid encoding BDNF operably linked to a sequence loxP sequence operably linked to a polyadenylation sequence, and a regulatory sequence including an AGRP promoter operatively linked to an interference RNA sequence (miR-Bdnf) (SEQ ID No:9) operatively linked to WPRE (SEQ ID NO:8) operatively linked to a polyadenylation sequence.

FIG. 5 provides the mRNA sequence for human BDNF (SEQ ID NO:5) and the human BDNF amino acid sequence (SEQ ID NO:10).

FIG. 6 provides the mRNA sequence for human trkB (SEQ ID NO:6) and the human trkB amino acid sequence (SEQ ID NO: 11).

FIG. 7 provides the DNA sequence of human AGRP (SEQ ID NO:7) and the human AGRP amino acid sequence (SEQ ID NO: 12).

FIG. 8 provides the DNA sequence for woodchuck post-transcriptional regulatory element (WPRE) (SEQ ID NO:8).

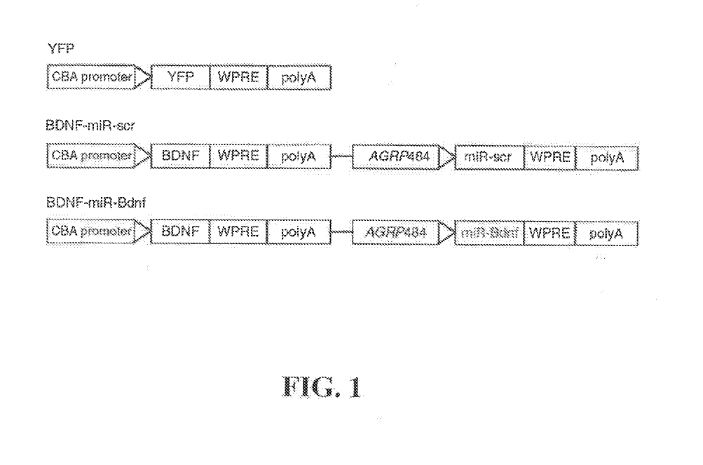

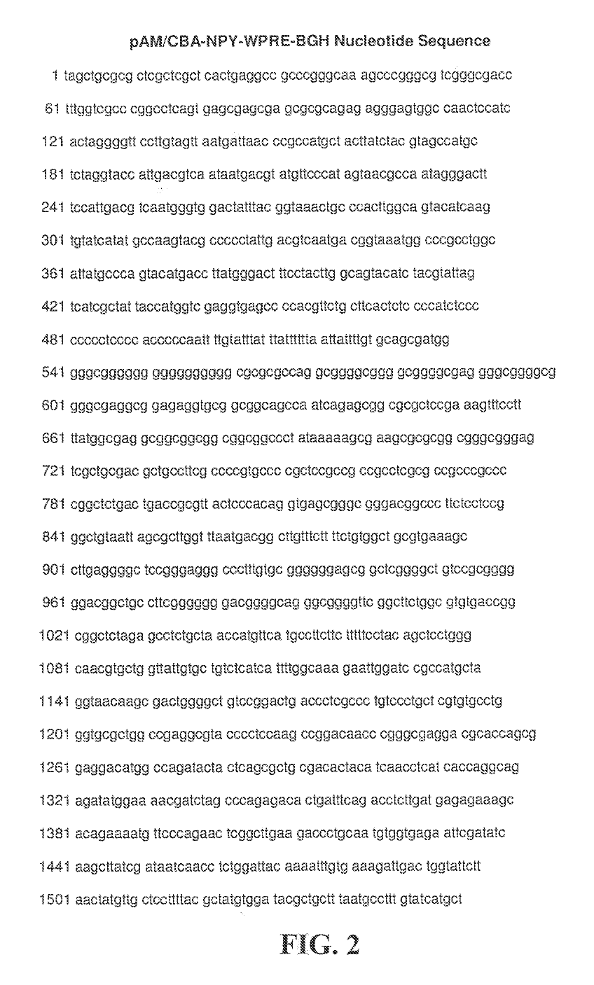

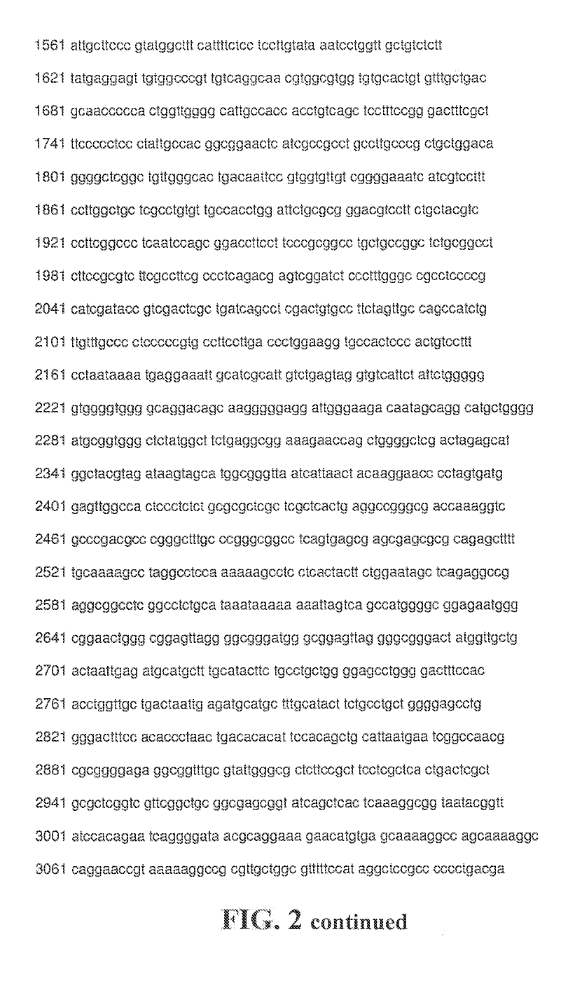

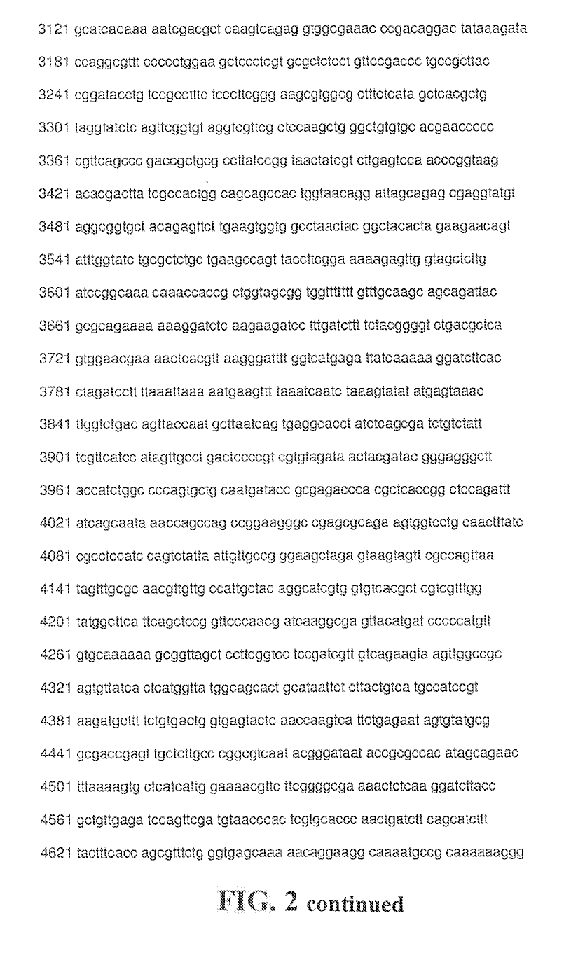

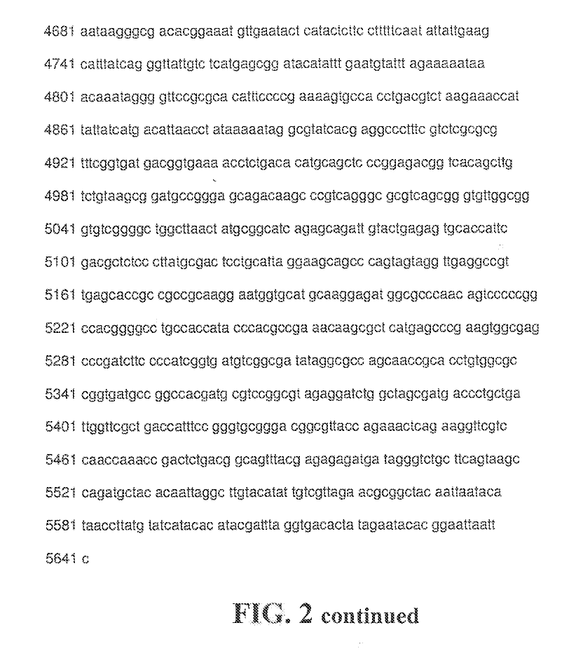

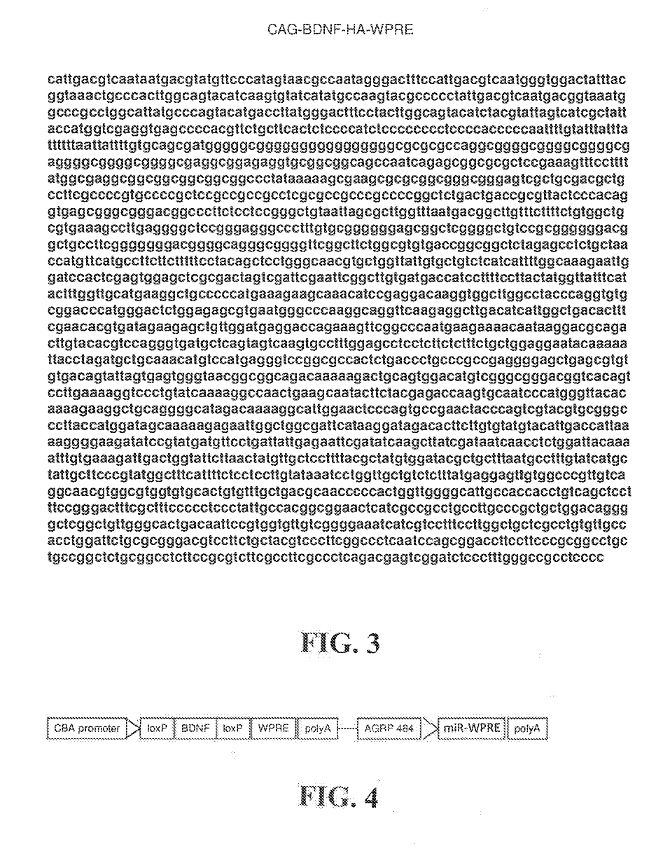

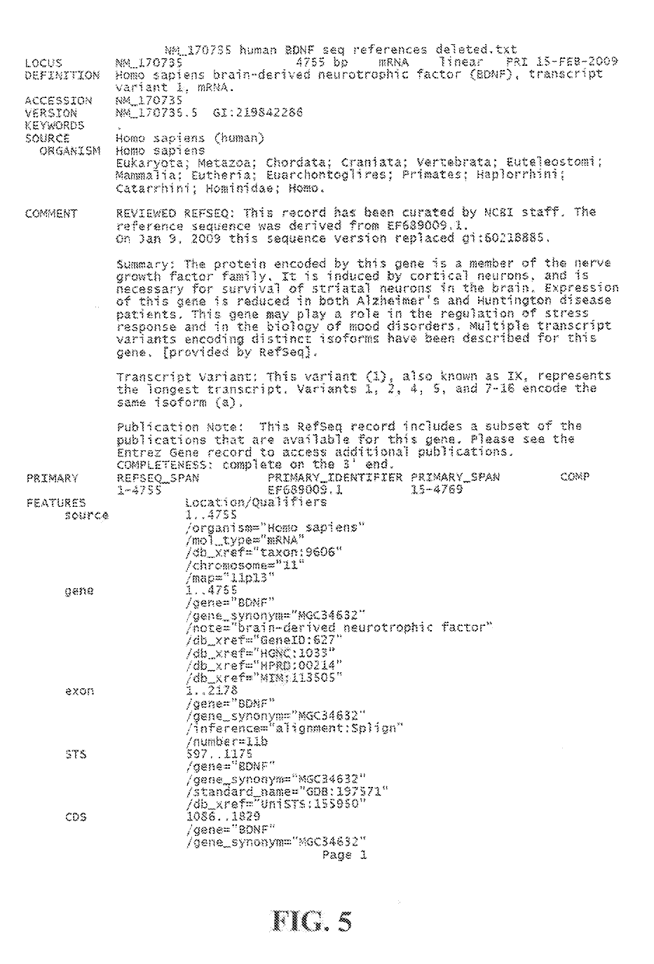

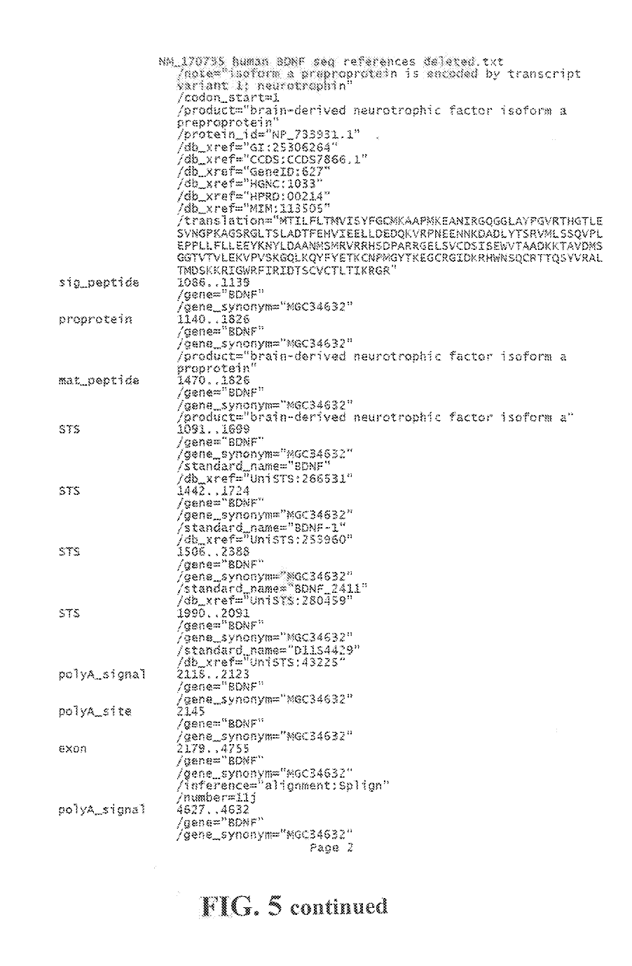

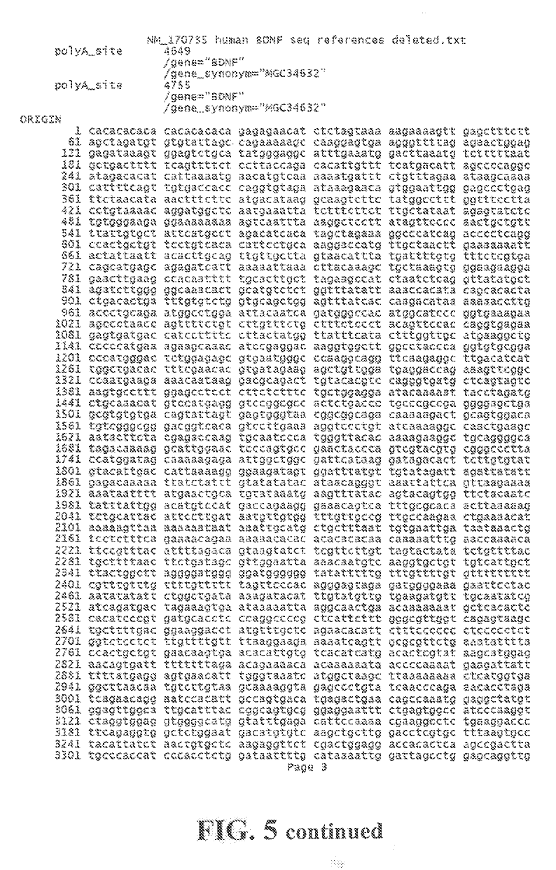

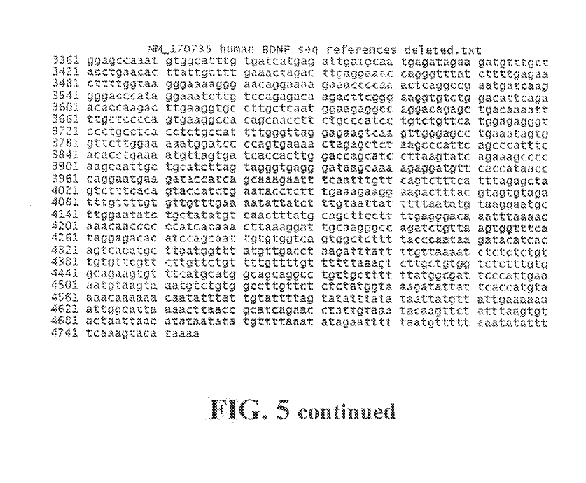

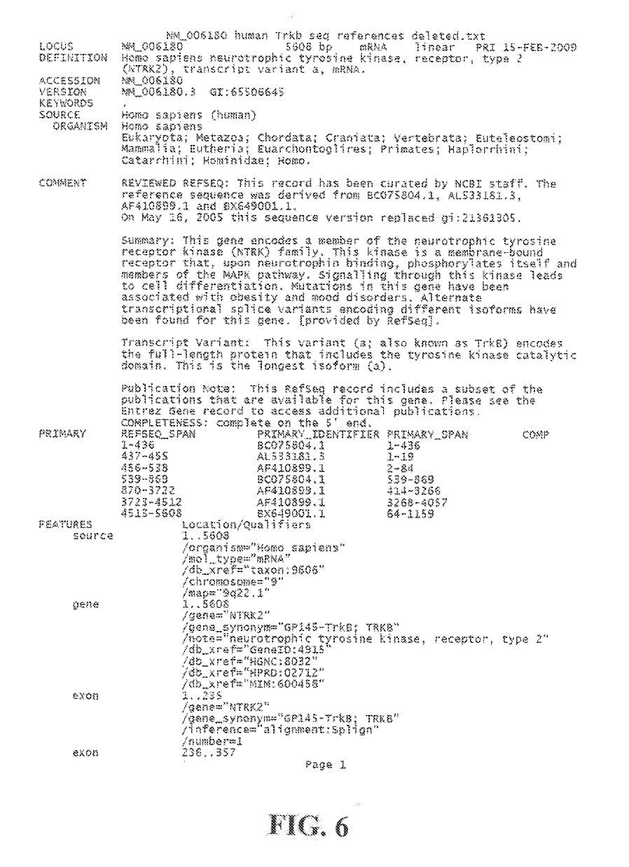

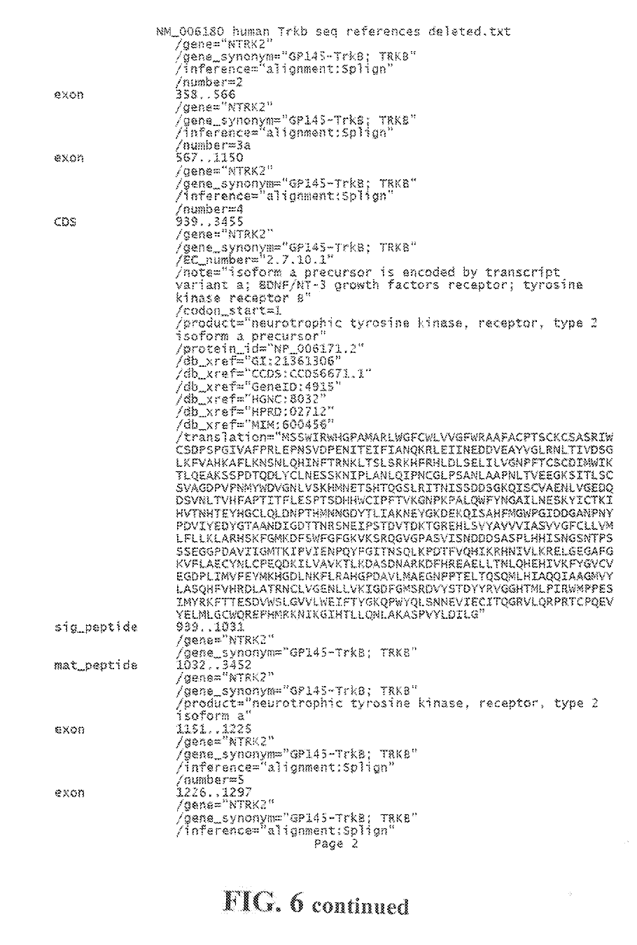

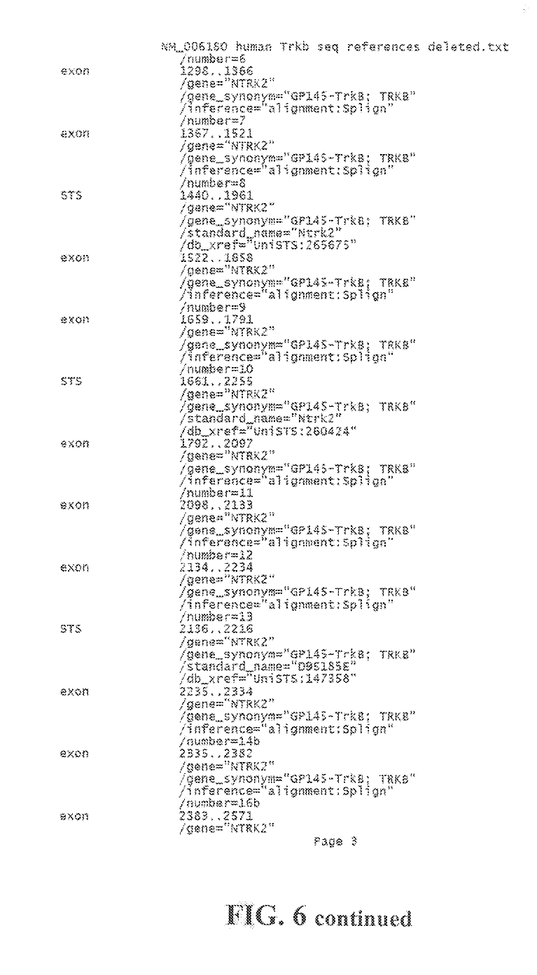

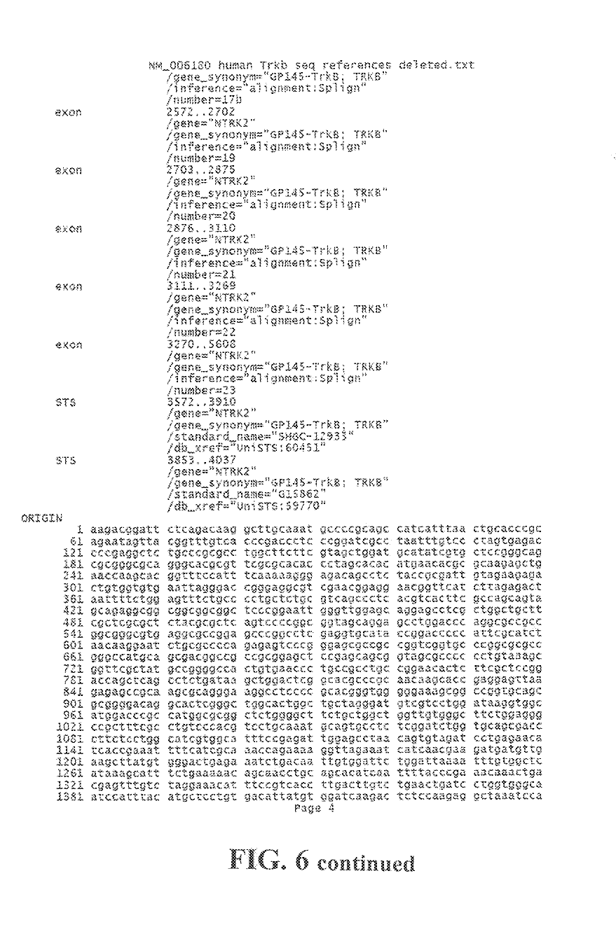

### DETAILED DESCRIPTION

Described herein are methods and compositions for treating a developmental syndrome with brain-derived neurotrophic factor (BNDF) delivered to one or more target areas in a patient's brain using a vector which causes expression of BDNF at the target area(s). In embodiments, the target area is a patient's hypothalamus. In embodiments, other areas of the brain may targeted. Any suitable vector known to those skilled in the art may be utilized to deliver BDNF to a target location in the brain. Upon such delivery, cells in the target locations are transfected or transductioned with nucleic acid encoding BDNF and can therefore be made to express BDNF.

Developmental syndromes suitable for treatment in accordance with the present disclosure include Prader-Willi syndrome, 16p11.2 deletion syndrome, 16p11.2 recurrent microdeletion, Albright hereditary osteodystrophy, Alström Syndrome, Bardet-Biedl syndrome, Borjeson-Forssman-Lehmann syndrome, Cohen syndrome, fragile X syndrome, fragile X syndrome (Prader-Willi Subtype), FMR1-Related Disorders, Down syndrome, Klinefelter syndrome, Turner syndrome, Smith-Magenis syndrome, Angelman syndrome, 21-Hydroxylase-Deficient Congenital Adrenal Hyperplasia, 2q37 Microdeletion syndrome, 3q29 Microdeletion syndrome, Achondroplasia, ADNP Syndrome, Melanocortin 4 receptor (MC4R) deficiency, Melanocortin 4 receptor (MC4R) disorder, Proopiomelanocortin Deficiency, 15q24 Microdeletion Syndrome, 15q Duplication Syndrome and Related Disorders, 1p36 Deletion Syndrome, and KANSL1-Related Intellectual Disability Syndrome. In embodiments, developmental syndromes herein manifest hypothalamic dysphasia. In embodiments, the developmental syndrome is melanocortin 4 receptor (MC4R) disorder.

In embodiments, a vector is utilized which includes one or more of: i) a nucleotide sequence encoding at least one neurotrophin such as BNDF that binds to a receptor and is capable of being delivered to a subject in need thereof for therapy of a developmental syndrome; ii) a nucleotide sequence encoding at least one receptor for the neurotrophin such as BNDF that binds to a receptor and is capable of being delivered to a subject in need thereof for therapy of developmental syndrome; and iii) a nucleotide sequence that mediates or facilitates the signaling of at least one neurotrophin that binds to a receptor and is capable of being delivered to a subject in need thereof for therapy of a developmental syndrome. In embodiments, the receptor is a trkB receptor capable of transducing one or more of the neurotrophin's effects.

In embodiments, a vector herein includes a combination of a BDNF transgene with a physiologically regulated RNA to that same transgene, or with a transgene mRNA including all untranslated 3′ and 5′ sequences, the vector capable of being delivered to a subject in need thereof for therapy of a developmental syndrome.

In embodiments, a vector herein may include a nucleotide sequence containing an expression cassette having an enhancer and promoter, and a regulatory gene sequence, wherein the nucleotide sequence encodes BDNF, a derivative or functional fragment thereof, and wherein the nucleotide sequence is inserted to one or more cloning sites between the promoter and the regulatory sequence.

In embodiments, the vector may be a stable integrating vector or a stable nonintegrating vector. In embodiments, the vector may be an adeno-associated viral vector (AAV), lentiviral vector or adenoviral vector. Lentiviruses are a subclass of retroviruses. Lentiviruses can integrate into the genome of non-dividing cells such as neurons. Lentiviruses are characterized by high-efficiency infection, long-term stable expression of transgenes and low immunogenicity.

AAV is a defective parvovirus known to infect many cell types and is nonpathogenic to humans. AAV can infect both dividing and non-dividing cells. Any of the known adeno-associated viruses may be utilized herein. In embodiments, the vector may be an adeno-associated viral vector selected from the serotype of one or more of: AAV-1, AAV-2, AAV-3, AAV-4, AAAV-5, AAV-6, AAV-7, AAV-8, AAV-9 and AAV-10. In embodiments, AAV1, AAV2, AAV4, AAV5, AAV8 and AAV9 may be utilized in connection with neurons. In embodiments, the vector may be any human or non-human primate isolate, variant, recombinant, chimeric or AAV capsid, including mutations, substitutions, deletions or additions. In embodiments, the adeno-associated viral vector may be AAV-2, or a modified form of AAV-2 with an altered tropism. In embodiments, the AAV nucleotide sequences may be derived from AAV serotype 1 (AAV-1). Additional suitable AAV serotypes have been developed through pseudotyping, i.e., mixing the capsid and genome from different viral serotypes. Accordingly, e.g., AAV2/7 indicates a virus containing the genome of serotype 2 packaged in the capsid from serotype 7. Other examples are AAV2/5, AAV2/8, AAV2/9, etc.

In embodiments, the vector is a recombinant adeno-associated virus (rAAV) virion containing a vector expression cassette having an enhancer and promoter, a regulatory gene sequence and a poly-A sequence flanked by AAV inverted terminal repeats (ITR), and having a biologically active protein cDNA fused at the 5′ terminal. The nucleotide sequences of AAV ITR regions are known. The AAV ITRs are regions found at each end of the AAV genome which function together in cis as origins of DNA replication and as packaging signals for the viral genome. AAV ITRs, together with the AAV rep coding region, provide for the efficient excision and rescue from, and integration of a nucleotide sequence interposed between two flanking ITRs into a mammalian cell genome. The ITR sequences for AAV-2 are described, for example by Kotin et al. (1994) Human Gene Therapy 5:793-801; Berns “Parvoviridae and their Replication” in Fundamental Virology, 2nd Edition, (B. N. Fields and D. M. Knipe, eds.) The AAV-2 ITR have 145 nucleotides. The terminal 125 nucleotides of each ITR form palindromic hairpin (HP) structures that serve as primers for AAV DNA replication. Each ITR also contains a stretch of 20 nucleotides, designated the D sequence, which is not involved in hairpin structure formation. Regions of the inverted terminal repeats (ITR) are designated as A, B, C, A′ and D at the 5′-end of the sequences and as D, A′, B/C, C/B and A at the 3′-end of the sequences. The site between these regions is referred to as the terminal resolution site, which serves as a cleavage site in the ITRs.

In embodiments, the AAV vector can be single stranded containing the ITRs which flank the genome. In embodiments, the AAV vector can be a double stranded so-called “self-complementary” (sc)AAV which also has ITRs flanking the genome by one ITR which is altered. In embodiments, there can be a deletion in the D-region of one of the ITRs which prevents rep-mediated nicking of the newly synthesized rAAV genome enabling efficient production and packaging of dimeric, double-stranded rAAV genomes into recombinant sc particles. The rAAV virion may be inserted to one or more cloning sites between the promoter and the regulatory sequence.

In embodiments, the biologically active protein is encoded by a BDNF nucleic acid sequence, or a derivative or functional fragment thereof, that is expressed in a target cell either constitutively or under regulatable conditions. In embodiments, the biologically active protein is a human BDNF protein sequence.

In embodiments, an autoregulatory system is utilized which may include a single rAAV vector harboring two expression cassettes, one constitutively driving BDNF, the other an interference RNA (RNAi) such as microRNA (miRNA), short hairpin RNA (shRNA) or short interference RNA (siRNA), targeting the BDNF gene, the RNAi being controlled by a promoter responsive to BDNF-induced physiological changes. Accordingly, as symptoms are reduced, the interference RNA becomes activated, thus inhibiting transgene expression and avoiding or preventing adverse effects which may be associated with non-regulated overexpression of BDNF.

In embodiments, the promoter of the BDNF transgene is a constitutive promoter. In embodiments, cellular or hybrid promoters which may also be responsive to the pathophysiological state can be used. In embodiments, when relief from symptoms of the developmental disorder is reached, the physiological responsive promoter might be stronger than the promoter driving the transgene. When the transgene overexpression leads to physiological changes, the weaker promoter controlling interfering RNA expression will be activated and thereby induce RNAi to inhibit the transgene expression. This system can provide a physiological negative feedback for all gene transfer studies and application in vivo and/or in vitro.

Adeno-associated viral vectors (AAV) can be constructed using known techniques to provide at least the operatively linked components of control elements including a transcriptional initiation region, an exogenous nucleic acid molecule, a transcriptional termination region and at least one post-transcriptional regulatory sequence. The control elements are selected to be functional in the targeted cell. The resulting construct which contains the operatively linked components is flanked at the 5′ and 3′ region with functional AAV ITR sequences. Indeed, suitable vectors may be constructed by those having ordinary skill in the art using known techniques. Suitable vectors can be chosen or constructed, containing, in addition to genes encoding BDNF and/or RNAi, appropriate regulatory sequences, including promoter sequences, terminator fragments, polyadenylation sequences, marker genes and other sequences as appropriate. Those skilled in the art are familiar with appropriate regulatory sequences, including promoter sequences, terminator fragments, polyadenylation sequences, marker genes and other suitable sequences. Typically, the vector includes a promoter to facilitate expression of the transgene within the target cell. In embodiments, specificity can be achieved by regional and cell-type specific expression of the receptor exclusively, e.g., using a tissue or region specific promoter. Virus gene promoter elements may help dictate the type of cells that are made to express BDNF. Some promoters are nonspecific (e.g., CAG, a synthetic promoter), while others are neuronal-specific (e.g., synapsin; hSyn), or preferential to specific neuron types, e.g., dynorphin, encephalin, GFAP (Glial fibrillary acidic protein) which is preferential to astrocytes, or CaMKIIa, which is preferential to cortical glutamatergic cells but can also target subcortical GABAergic cells. In embodiments, the promoter may be a Camklla (alpha CaM kinase II gene) promoter, which may drive expression in the forebrain. Other neuronal cell type-specific promoters include the NSE promoter, tyrosine hydroxylase promoter, myelin basic protein promoter, glial fibrillary acidic protein promoter, and neurofilaments gene (heavy, medium, light) promoters.

In embodiments, the inducible regulatory sequence renders BDNF expression central nervous system-specific. In embodiments, the target cell is a mammalian cell. In embodiments, the target cell is a human cell. In embodiments, the target cell is in cell culture. In embodiments, the target cell is in a living mammal.

An AAV vector harboring the BDNF transgene flanked by AAV ITRs, can be constructed, e.g., by directly inserting the transgene into an AAV genome which has had the major AAV open reading frames (“ORFs”) excised therefrom. Other portions of the AAV genome can also be deleted, as long as a sufficient portion of the ITRs remain to allow for replication and packaging functions. These constructs can be designed using techniques well known in the art.

Vectors herein may contain reporter genes, e.g., those which encode fluorophores. A fluorophore is a fluorescent compound that can re-emit light upon excitation, usually at specific frequencies. They can be used as a tag or marker which can be attached to, e.g., a protein to allow the protein to be located. Many suitable fluorophores are known in the art. They may be categorized by the color they emit, e.g., blue, cyan, green, yellow, orange, red and others. For example, mCherry, mRasberry, mTomato and mRuby are red fluorophore proteins; citrine, venus, and EYFP are yellow fluorophore proteins.

In embodiments, the vector may include a nucleotide sequence represented by SEQ ID NO: 1. In embodiments, the nucleotide sequence may include the DNA sequence represented by AGRP484 (484 bp, −133 bp to +351 bp from the start of the noncoding exon28) in SEQ ID NO:7. In embodiments, the nucleotide sequence includes the DNA sequence represented by AGRP814 (814 bp, −463/+351) in SEQ ID NO:7. AGRP484 and AGRP814 are promoters responsive to BDNF-induced physiological changes. See, e.g., Cao et al., supra.

In embodiments, an AAV vector is provided which retains only the replication and packaging signals of AAV, and which includes a nucleotide sequence encoding BDNF, or a derivative or a functional fragment thereof. In embodiments, an AAV vector is provided, wherein the nucleic acid sequence comprises a nucleic acid sequence of SEQ ID NO: 1, AGRP484 (484 bp, −133 bp to +351 bp from the start of the noncoding exon28) in SEQ ID NO:7 or AGRP814 (814 bp, −463/+351) in SEQ ID NO:7, or a derivative or a functional fragment thereof.

In embodiments, a vector expression cassette includes a promoter selected from: chicken ß-actin (CBA), agouti related protein 484 (AGRP484), and agouti related protein 814 (AGRP814).

In embodiments, an rAAV vector includes a vector expression cassette including an enhancer, a promoter, a regulatory element and bovine growth hormone polyadenosine flanked by AAV inverted terminal repeats, wherein fused human BDNF cDNA is fused at the 5′ terminus and then inserted into at least one multiple cloning site between the promoter and the sequence.

In embodiments, an rAAV vector includes a vector expression cassette including a cytomegalovirus enhancer, a chicken ß-actin (CBA) promoter, a woodchuck post-transcriptional regulatory element (WPRE) and bovine growth hormone polyadenosine flanked by AAV inverted terminal repeats, wherein fused human BDNF cDNA is fused at the 5′ terminus and then inserted into multiple cloning sites between the CBA promoter and the WPRE sequence.

In embodiments, the rAAV vector includes a weaker promoter to drive the BDNF, wherein, in a pre-therapy state the AGRP promoter is dialed down, but is activated when BDNF is overexpressed resulting in undesirable symptoms such as exaggerated weight loss, hyperexcitability, anxiety, and wherein, at desirable symptom management, the AGRP promoter is stronger than the promoter driving the BDNF. Activation of the AGRP promoter operatively linked to the RNAi induces expression of the RNAi which reduces BDNF expression, thereby reducing or alleviating undesirable symptoms of BDNF overexpression.

In embodiments, a vector incorporating the BDNF transgene may incorporate a knock-out feature, thereby rendering BDNF expression in transfected or transductioned cells inoperable. For example, a loxP-Cre recombination system can be utilized. A rAAV vector constructed with the BDNF transgene flanked by two loxP sites (flox-BDNF), can be subsequently knocked out by a second viral vector delivering Cre recombinase. The rAAV vector encoding a GFP-Cre fusion protein has been shown to efficiently ablate loxP-modified genes in the brain, including the hypothalamus, with low toxicity. Those skilled in the art are familiar with loxP-Cre recombination systems and techniques for incorporating them into vectors.

In embodiments, provided herein is a method for ameliorating a symptom of a developmental syndrome in a mammal, the method comprising direct administration of an adeno-associated virus-derived vector to a target cell in the brain of the mammal, the vector comprising a DNA sequence, wherein the DNA sequence is exogenous to an adeno-associated virus and comprises a sequence encoding a therapeutic protein such as BDNF in operable linkage with a promoter sequence, wherein the adeno-associated virus-derived vector is free of both wild-type and helper virus, and wherein the exogenous DNA sequence is expressed in the target cell such that the symptom of the developmental syndrome is ameliorated.

In embodiments, a method for treating a mammal with a developmental syndrome is provided which includes administering an expression vector to a target cell in the mammal, wherein the expression vector includes a nucleic acid sequence encoding BDNF, or a derivative or functional fragment thereof, and wherein the administering results in expression of BDNF, or a derivative or functional fragment thereof, in the target cell and the expression reduces one or more symptoms of the developmental syndrome, thereby treating the mammal with such syndrome. In embodiments, the expression vector is a viral or a non-viral expression vector. In embodiments, the viral expression vector is an adeno-associated virus (AAV) vector. In embodiments, the nucleic acid sequence encoding BDNF is a nucleic acid sequence encoding an amino acid sequence according to SEQ ID NO: 1, AGRP484 (484 bp, −133 bp to +351 bp from the start of the noncoding exon28) in SEQ ID NO:7 or AGRP814 (814 bp, −463/+351) in SEQ ID NO:7, or a derivative or a functional fragment thereof. In embodiments, the developmental syndrome is melanocortin 4 receptor (MC4R) disorder. In embodiments, the administering is by stereotaxic microinjection.

In embodiments, there is provided herein a method for delivering a nucleotide sequence such as nucleic acid encoding BDNF to a mammalian nervous system target cell, the method comprising administering an adeno-associated virus (AAV) vector to the target cell, wherein the vector transductions the target cell; and wherein the AAV vector is free of both wildtype and helper virus.

The vectors carrying the nucleic acid encoding at least one heterologous protein such as BDNF can be precisely delivered into specific sites of the central nervous system and the brain, using stereotactic microinjection techniques. For example, the subject being treated can be placed within a stereotactic frame base (MRI-compatible) and then imaged using high resolution MRI to determine the three-dimensional positioning of the particular region to be treated. The MRI images can then be transferred to a computer having the appropriate stereotactic software, and a number of images are used to determine a target site and trajectory for pharmacological agent microinjection. The software translates the trajectory into three-dimensional coordinates that are precisely registered for the stereotactic frame. In the case of intracranial delivery, the skull will be exposed, burr holes will be drilled above the entry site, and the stereotactic apparatus used to position the needle and ensure implantation at a predetermined depth. The pharmacological agent can be delivered to regions, such as the cells of the spinal cord, brainstem, or brain that are associated with the syndrome. For example, target regions can include the medulla, pons, and midbrain, cerebellum, diencephalons (e.g., thalamus, hypothalamus), telencephalon (e.g., corpus stratium, cerebral cortex, or within the cortex, the occipital, temporal, parietal or frontal lobes), or combinations thereof.

In embodiments, a route of delivery is an approach via the ventricles, with or without an endoscope. In embodiments, the vector may be delivered via the lateral ventricle, through to the third ventricle which lies immediately adjacent to the hypothalamus. Another approach is transnasally, such as transphenoidally, involving a direct approach through the nasal sinuses to the base of the brain, and then a delivery device inserted to deliver the vector directly through this skull base approach into the hypothalamus. Still another approach can be to deliver the vector simply into the ventricles with sufficient hypothalamic expression obtained to induce amelioration of symptoms of a developmental syndrome. As discussed below, ultrasound treatment may be utilized during delivery.

As used herein, the terms “gene transfer,” “gene delivery,” and “gene transduction” can refer to methods or systems for reliably inserting a particular nucleotide sequence (e.g., DNA) into targeted cells. As used herein, the term “gene therapy” can refer to a method of treating a patient wherein polypeptides or nucleic acid sequences are transferred into cells of a patient such that activity and/or the expression of a particular molecule is restored.

As used herein, the term “gene” refers to an assembly of nucleotides that encodes a polypeptide and includes cDNA and genomic DNA nucleic acids. A gene is a nucleic acid that does not necessarily correspond to the naturally occurring gene which contains all of the introns and regulatory sequences, e.g., promoters, present in the natural genomic DNA. Rather, a gene encoding a particular protein can minimally contain just the corresponding coding sequence for the protein.

As used herein, a “promoter sequence” is a DNA regulatory region capable of binding RNA polymerase in a cell and initiating transcription of a downstream (3′ direction) coding sequence. The promoter sequence is typically bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

As used herein, transcriptional and translational control sequences are DNA regulatory sequences, such as promoters, enhancers, terminators, and the like, that provide for the expression of a coding sequence in a host cell. In eukaryotic cells, polyadenylation signals are control sequences.

An “expression control sequence” is a DNA sequence that controls and regulates the transcription and translation of another DNA sequence. A coding sequence is “operatively under the control” of transcriptional and translational control sequences in a cell when RNA polymerase transcribes the coding sequence into a precursor RNA, which is then trans-RNA spliced to yield mRNA and translated into the protein encoded by the coding sequence.

A nucleotide sequence is “operatively under the control” of a genetic regulatory sequence when the genetic regulatory sequence controls and/or regulates the transcription of that nucleotide sequence. That genetic regulatory sequence can also be referred to as being “operatively linked” to that nucleotide sequence.

As used herein, a “genetic regulatory sequence” is a nucleic acid that: (a) acts in cis to control and/or regulate the transcription of a nucleotide sequence, and (b) can be acted upon in trans by a regulatory stimulus to promote and/or inhibit the transcription of the nucleotide sequence. Therefore, an inducible promoter is a genetic regulatory sequence. In addition, a portion of a promoter (e.g., fragment/element) that retains and/or possesses the ability to control and/or regulate the transcription of a nucleotide sequence either alone or in conjunction with an alternative promoter or fragment thereof (e.g., a chimeric promoter), is also a genetic regulatory sequence. Such fragments include response elements (genetic response elements) and promoter elements.

As used herein, an “expression cassette” is a nucleic acid that minimally comprises a nucleotide sequence to be transcribed (e.g., a coding sequence) that is operatively under the control of a genetic regulatory sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

As used herein, a “heterologous gene” is a gene that has been placed into a vector or cell that does not naturally occur in that vector or cell.

As used herein, a gene is an “exogenous gene” when the gene is not encoded by the particular vector or cell.

A “vector” as used herein is a genetic construct that facilitates the efficient transfer of a nucleic acid (e.g., a gene) to a cell. The use of a vector can also facilitate the transcription and/or expression of that nucleic acid in that cell. Non-limiting examples of vectors include plasmids, phages, amplicons, viruses and cosmids, to which another DNA segment may be attached so as to bring about the replication of the attached segment. The vectors can be any vector suitable for delivering the nucleic acid encoding a heterologous protein to a host cell at the target site. The term “vector” as used herein refers to any genetic element, such as a plasmid, phage, transposon, cosmid, chromosome, artificial chromosome, virus, virion, non-viral vectors including polymers, liposomes and various non-viral chemical complexes, and the like. Thus, the term includes cloning and expression vehicles, as well as viral vectors.

The term “subject” as used herein refers to any living organism in which an immune response is elicited. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered. “Subject” and “patient” are used interchangeably herein.

The term “mammal” as used herein refers to a living organism capable of eliciting a humoral immune response to an antigen. The term subject includes, but is not limited to, nonhuman primates such as chimpanzees and other apes and monkey species, sheep, pigs, goats, horses, dogs, cats, mice, rats and guinea pigs, and the like.

In embodiments, methods and compositions for treating a developmental syndrome include applying ultrasound to a target location in the patient's brain to enhance permeability of the patient's blood brain barrier at the target location and administering to the patient a vector encoding BDNF for delivery of the BDNF to the target location. It should be understood that any vector suitable for transfecting, transductioning or transducing brain cells with nucleic acid encoding BDNF including any which have been discussed previously herein may be utilized. Once a determination has been made of the location or of a suspected location of an area associated with a developmental syndrome in a patient, targeted treatment in accordance with the present disclosure can be implemented. For example, such areas include, but are not limited to, the hypothalamus, the thalamus, the cerebellum, the cerebrum, the medulla, the pons, the midbrain, the temporal lobe, the frontal lobe, the occipital lobe and the parietal lobe.

Use of focused ultrasound energy herein disrupts the BBB without adversely affecting the vector and/or brain tissue itself. This may be considered surprising in view of potential damage to organic compounds and tissues by ultrasound energy. Use of ultrasound energy herein can increase the speed of delivery of vectors to target locations in the brain, reduce side effects which may be associated with delivery of vectors to target locations in the brain, reduce dosage amounts while concentrating vectors at a target location and can allow controlled release of the amount of vectors at a target location.

In accordance with the present disclosure, in embodiments, ultrasound energy assists and/or propels penetration of the vector carrying BDNF to target locations in the brain. In embodiments, ultrasound energy is used to make the blood brain barrier permeable to vectors herein. Accordingly, in embodiments, ultrasound energy can be applied to a target location prior to administration of the vector. In embodiments, vectors herein can be administered to a target area in the brain simultaneously with administration of ultrasound energy. In embodiments, vectors herein can be administered to a target area in the brain after administration of ultrasound energy. In embodiments, vectors herein can be administered systemically. Systemic delivery includes oral, buccal, sublingual, rectal, topical, intranasal, vaginal and parenteral modes of administration. Examples of parenteral modes of administration include intravenous, intraperitoneal, intramuscular and subcutaneous modes of administration. In this manner, vectors circulating in the blood stream are delivered to a target location in the brain through a portion of the BBB disrupted by ultrasonic energy. In embodiments, vectors herein can be administered systemically after ultrasound energy treatment of the target location and the vectors penetrate the disrupted BBB to become situated at the target location. In embodiments, vectors herein can be administered directly to a target location in the brain. In embodiments, vectors herein can be administered directly to a target location in the brain after ultrasound energy treatment of the target location to become situated at the target location. In embodiments, vectors herein can be administered directly to a target location in the brain without ultrasound treatment.

Methods for administering materials directly to target locations within the brain are well-known. For example, a hole can be drilled into the skull and an appropriately sized needle may be used to deliver a vector to a target location. In embodiments, a portion of the skull may be removed to expose the dura matter (craniotomy) at or near a target location and a vector can be administered directly to the target location with or without ultrasound energy. In embodiments, a vector is injected intracranially using stereotaxic coordinates, a micropipette and an automated pump for precise delivery of the vector to the desired area with minimal damage to the surrounding tissue. In embodiments, a micropump may be utilized to deliver pharmaceutical compositions containing a vector to target areas in the brain. The compositions can be delivered immediately or over an extended period of time, e.g., over 1, 2, 3, 4, 5, 6, 7, 8, 9, 10 or more minutes. After vector delivery of the vector to a target location in the brain a sufficient amount of time may be allowed to pass to allow expression of BDNF at the target location. Thus, the vector can be administered directly to a target location in the brain by any known means for administering materials to the brain, e.g., direct injection.

In embodiments, ultrasound energy can be administered to a target area by removing a portion of the skull (craniotomy) to expose the dura matter at or near a target location and delivering the ultrasound energy at or below the exposed dura matter. In embodiments, ultrasound energy can be administered to a target location through the skull, eliminating the need for surgery associated with delivery of ultrasound energy to a target location. Methods for delivering ultrasound energy through the skull are known in the art. See, e.g., U.S. Pat. No. 5,752,515 and US Publication No. 2009/0005711, both of which are hereby incorporated by reference in their respective entireties. See also, Hynynen et al., NeuroImage 24 (2005) 12-120.

In embodiments, ultrasound energy can be applied to a target location in the brain at frequencies ranging from about 20 kHz to about 5 MHz, and with sonication duration ranging from 100 nanoseconds to 1 minute. In embodiments, ultrasound energy can be applied to a target location in the brain at frequencies ranging from about 20 kHz to about 10 MHz, sonication duration ranging from about 100 nanoseconds to about 30 minutes, with continuous wave or burst mode operation, where the burst mode repetition varies from about 0.01 Hz to about 1 MHz. In embodiments, ultrasound energy can be applied to a target location in the brain at frequencies ranging from about 200 kHz to about 10 MHz, and with sonication duration ranging from about 100 milliseconds to about 30 minutes. In embodiments, ultrasound energy can be applied to a target location in the brain at frequencies ranging from about 250 kHz to about 10 MHz, and with sonication duration ranging from about 0.10 microseconds to about 30 minutes. In embodiments, ultrasound energy can be applied to a target location in the brain at a frequency of about 1.525 MHz. In embodiments, ultrasound energy can be applied to a target location in the brain at a frequency of about 0.69 MHz. In embodiments, pressure amplitudes generated by ultrasound energy can be about 0.5 to about 2.7 MPa. In embodiments, pressure amplitudes generated by ultrasound energy can be about 0.8 to about 1 MPa. In embodiments, ultrasound energy is applied to a target location in the brain at a focal region sized in accord with the volume of tissue and/or fluids to which a vector or synthetic ligand is to be delivered, e.g., from about 0.1 mm³ to about 5 cm³.

In embodiments, the target location and access thereto is confirmed by introducing a contrast agent into the patient prior to, during or after application of ultrasound energy to the target location, allowing sufficient time for the contrast agent to permeate the BBB, and determining whether the contrast agent is present at the target location. Contrast agents are well-known and include, e.g., iodine-based compounds, barium-based compounds and lanthanide based compounds. Iodine-based agents include, e.g., iohexol, iopromide, iodixanol, iosimenol, ioxaglate, iothalamate and iopamidol. Barium-based compounds include barium sulfate. Lanthanide-based compounds include, e.g., gadolinium-based chelates such as gadoversetamide, gadopentetate dimeglumine, gadobutrol, gadobenate dimeglumine, gadoterate meglumine, and gadoxetate disodium. Detection modalities include 2-dimensional X-ray radiography, X-ray computed tomography and magnetic resonance imaging which are well-known techniques that may be utilized to confirm the presence or absence of contrast agent in a target location.

In embodiments, provided herein are methods for treating a developmental syndrome which include applying ultrasound to a target location in the patient's brain to enhance permeability of the patient's blood brain barrier at the target location; administering to the patient a vector containing nucleic acid encoding BDNF for delivery of the BDNF to the target location, wherein the patient exhibits improvement in at least one symptom of the developmental syndrome. In embodiments, provided herein are methods for treating a developmental syndrome which include applying ultrasound to a target location in the patient's brain to enhance permeability of the patient's blood brain barrier at the target location; administering to the patient a vector containing an autoregulatory system as described above which may include a single rAAV vector harboring two expression cassettes, one constitutively driving BDNF, the other an interference RNA (RNAi) such as microRNA (miRNA), short hairpin RNA (shRNA) or short interference RNA (siRNA), targeting the BDNF gene, the RNAi being controlled by a promoter responsive to BDNF-induced physiological changes.

In embodiments, methods of treating a developmental syndrome are provided which include applying ultrasound to a target location in the patient's brain to enhance permeability of the patient's blood brain barrier at the target location; administering to the patient a vector containing nucleic acid encoding BDNF for delivery of the BDNF to the target location, the vector also containing an autoregulatory sequence for reducing expression of BDNF; applying ultrasound to a target location in the patient's brain to enhance permeability of the patient's blood brain barrier at the target location; wherein the method provides improvement in one or more symptoms of the developmental syndrome for more than 1 hour after administration to the patient. In embodiments, the method provides improvement in one or more symptoms of the developmental syndrome for more than 2 hours after administration of the vector to the patient. In embodiments, the method provides improvement in one or more symptoms of the developmental syndrome for more than 3 hours after administration of the vector to the patient. In embodiments, the method provides improvement in one or more symptoms of the developmental syndrome for more than 4 hours after administration of the vector to the patient. In embodiments, the method provides improvement in one or more symptoms of the developmental syndrome for more than 6 hours after administration of the vector to the patient. In embodiments, the method provides improvement in one or more symptoms of the developmental syndrome for more than 8, 10, 12, 14, 16, 18, 20, 22 or 24 hours after administration of the vector to the patient. In embodiments, improvement in at least one symptom for 12 hours after administration of the vector to the patient is provided in accordance with the present disclosure. In embodiments, the methods provide improvement of next day functioning of the patient. For example, the method may provide improvement in one or more symptoms of the developmental syndrome for more than about, e.g., 2 hours, 4 hours, 6 hours, 8 hours, 10 hours, 12 hours, 14 hours, 16 hours, 18 hours, 20 hours, 22 hours or 24 hours after administration and waking from a night of sleep.

In embodiments, the methods described herein are effective to reduce, delay, or prevent one or more other clinical symptoms of a developmental syndrome. For example, the effect, in a patient having vector transformed cells that express BDNF in a target location of the brain, whose delivery is enhanced by ultrasound energy on a particular symptom, pharmacologic, or physiologic indicator can be compared to an untreated patient, or the condition of the patient prior to treatment. In embodiments, the symptom, pharmacologic, and/or physiologic indicator is measured in a patient prior to treatment, and again one or more times after treatment is initiated. In embodiments, the control is a reference level, or average determined based on measuring the symptom, pharmacologic, or physiologic indicator in one or more patients that do not have the disease or condition to be treated (e.g., healthy patients). In embodiments, the effect of the treatment is compared to a conventional treatment that is within the purview of those skilled in the art.

Effective treatment of a developmental syndrome herein may be established by showing reduction in the frequency or severity of symptoms (e.g., more than 10%, 20%, 30% 40% or 50%) after a period of time compared with baseline. For example, after a baseline period of 1 month, the patients having modified receptors may be randomly allocated a BDNF vector, or placebo as add-on therapy to standard therapies, during a double-blind period of 2 months. Primary outcome measurements may include the percentage of responders on a BDNF vector, and on placebo, defined as having experienced at least a 10% to 50% reduction of symptoms during the second month of the double-blind period compared with baseline.

In embodiments, pharmaceutical compositions containing vectors described herein may be prepared using a pharmaceutically acceptable “carrier” composed of materials that are considered safe and effective. The “carrier” includes all components present in the pharmaceutical formulation other than the active ingredient or ingredients. The term “carrier” includes, but is not limited to, diluents, binders, lubricants, disintegrants, fillers, and coating compositions. Those with skill in the art are familiar with such pharmaceutical carriers and methods of compounding pharmaceutical compositions using such carriers.

In embodiments, pharmaceutical compositions containing vectors are suitable for parenteral administration, including, e.g., intramuscular (i.m.), intravenous (i.v.), subcutaneous (s.c.), intraperitoneal (i.p.), or intrathecal (i.t.). Parenteral compositions must be sterile for administration by injection, infusion or implantation into the body and may be packaged in either single-dose or multi-dose containers. In embodiments, liquid pharmaceutical compositions for parenteral administration to a patient include an active substance, e.g., vectors. In embodiments, the pharmaceutical compositions for parenteral administration are formulated as a total volume of about, e.g., 0.1 ml, 0.25 ml, 0.5 ml, 0.75 ml, 1 ml, 1.25 ml, 1.5 ml, 1.75 ml, 2 ml, 2.25 ml, 2.5 ml, 2.75 ml, 3 ml, 3.25 ml, 3.5 ml, 3.75 ml, 4 ml, 4.25 ml, 4.5 ml, 4.75 ml, 5 ml, 10 ml, 20 ml, 25 ml, 50 ml, 100 ml, 200 ml, 250 ml, or 500 ml. In embodiments, the volume of pharmaceutical compositions containing vectors are microliter amounts. For example, 0.1 microliters to 10 or more microliters can be injected. For example, 0.1, 0.2, 0.3, 0.4, 0.5, 0.6, 0.7, 0.8, 0.9, 1.0, 1.25, 1.5, 1.75, 2.0, 2.25, 2.5, 2.75, 3.0, 3.25, 3.5, 3.75, 4.0, 4.25, 4.5, 4.75, 5.0, 5.25, 5.5, 5.75, 6.0, 6.25, 6.5, 6.75, 7.0, 8.25, 8.5, 8.75, 9.0, 9.25, 9.5, 9.75, or 10 microliters. In embodiments, the compositions are contained in a micropipette, a bag, a glass vial, a plastic vial, or a bottle.

In embodiments, pharmaceutical compositions for parenteral administration may include about 0.0001 mg to about 500 mg active substance, e.g., vectors. In embodiments, pharmaceutical compositions for parenteral administration to a patient include an active substance, e.g., vectors at a respective concentration of about 0.001 mg/ml to about 500 mg/ml. In embodiments, the pharmaceutical composition for parenteral administration includes an active substance, e.g., vector at a concentration of, e.g., about 0.005 mg/ml to about 50 mg/ml, about 0.01 mg/ml to about 50 mg/ml, about 0.1 mg/ml to about 10 mg/ml, about 0.05 mg/ml to about 25 mg/ml, about 0.05 mg/ml to about 10 mg/ml, about 0.05 mg/ml to about 5 mg/ml, or about 0.05 mg/ml to about 1 mg/ml. In embodiments, the pharmaceutical composition for parenteral administration includes an active substance, e.g., vector, at a concentration of, e.g., about 0.05 mg/ml to about 15 mg/ml, about 0.5 mg/ml to about 10 mg/ml, about 0.25 mg/ml to about 5 mg/ml, about 0.5 mg/ml to about 7 mg/ml, about 1 mg/ml to about 10 mg/ml, about 5 mg/ml to about 10 mg/ml, or about 5 mg/ml to about 15 mg/ml.

In embodiments, a pharmaceutical composition for parenteral administration is provided wherein the pharmaceutical composition is stable for at least six months. In embodiments, the pharmaceutical compositions for parenteral administration exhibit no more than about 5% decrease in active substance, e.g., vector, for at least, e.g., 3 months or 6 months. In embodiments, the amount of vector degrades at no more than about, e.g., 2.5%, 1%, 0.5% or 0.1%. In embodiments, the degradation is less than about, e.g., 5%, 2.5%, 1%, 0.5%, 0.25%, 0.1%, for at least six months.

In embodiments, pharmaceutical compositions for parenteral administration are provided wherein the pharmaceutical composition remains soluble. In embodiments, pharmaceutical compositions for parenteral administration are provided that are stable, soluble, local site compatible and/or ready-to-use. In embodiments, the pharmaceutical compositions herein are ready-to-use for direct administration to a patient in need thereof.

The pharmaceutical compositions for parenteral administration provided herein may include one or more excipients, e.g., solvents, solubility enhancers, suspending agents, buffering agents, isotonicity agents, stabilizers or antimicrobial preservatives. When used, the excipients of the parenteral compositions will not adversely affect the stability, bioavailability, safety, and/or efficacy of a vector used in the composition. Thus, parenteral compositions are provided wherein there is no incompatibility between any of the components of the dosage form.

In embodiments, parenteral compositions including a vector include a stabilizing amount of at least one excipient. For example, excipients may be selected from the group consisting of buffering agents, solubilizing agents, tonicity agents, antioxidants, chelating agents, antimicrobial agents, and preservative. One skilled in the art will appreciate that an excipient may have more than one function and be classified in one or more defined group.

In embodiments, parenteral compositions include a vector and an excipient wherein the excipient is present at a weight percent (w/v) of less than about, e.g., 10%, 5%, 2.5%, 1%, or 0.5%. In embodiments, the excipient is present at a weight percent between about, e.g., 1.0% to 10%, 10% to 25%, 15% to 35%, 0.5% to 5%, 0.001% to 1%, 0.01% to 1%, 0.1% to 1%, or 0.5% to 1%. In embodiments, the excipient is present at a weight percent between about, e.g., 0.001% to 1%, 0.01% to 1%, 1.0% to 5%, 10% to 15%, or 1% to 15%.

In embodiments, parenteral compositions containing a vector(s) herein may be administered as needed, e.g., once, twice, three, four, five, six or more times daily, or continuously depending on the patient's needs. In embodiments, parenteral compositions containing a vector(s) herein may be administered once and be effective for providing prolonged relief from symptoms of a developmental syndrome. In embodiments, parenteral compositions containing a vector(s) herein may be administered once a month, once every two months, once every three months, once every four months, once every six months or once every twelve months and still be effective in providing prolonged relief from symptoms of a developmental syndrome.

In embodiments, parenteral compositions of an active substance, e.g., a vector or a synthetic ligand or a pharmaceutically acceptable salt of the synthetic ligand, are provided, wherein the pH of the composition is between about 4.0 to about 8.0. In embodiments, the pH of the compositions is between, e.g., about 5.0 to about 8.0, about 6.0 to about 8.0, about 6.5 to about 8.0. In embodiments, the pH of the compositions is between, e.g., about 6.5 to about 7.5, about 7.0 to about 7.8, about 7.2 to about 7.8, or about 7.3 to about 7.6. In embodiments, the pH of the aqueous solution is, e.g., about 6.8, about 7.0, about 7.2, about 7.4, about 7.6, about 7.7, about 7.8, about 8.0, about 8.2, about 8.4, or about 8.6.

Unless defined otherwise, all technical and scientific terms used herein have the same meanings as commonly understood by one of skill in the art to which the disclosure herein belongs.

The term “about” or “approximately” as used herein means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and/or up to 1% of a given value. Alternatively, particularly with respect to biological systems or processes, the term can mean within an order of magnitude, preferably within 5-fold, and more preferably within 2-fold, of a value.

“Improvement” refers to the treatment of a developmental syndrome such as Prader-Willi syndrome, 16p11.2 deletion syndrome, 16p11.2 recurrent microdeletion, Albright hereditary osteodystrophy, Alström Syndrome, Bardet-Biedl syndrome, Borjeson-Forssman-Lehmann syndrome, Cohen syndrome, fragile X syndrome, fragile X syndrome (Prader-Willi Subtype), FMR1-Related Disorders, Down syndrome, Klinefelter syndrome, Turner syndrome, Smith-Magenis syndrome, Angelman syndrome, 21-Hydroxylase-Deficient Congenital Adrenal Hyperplasia, 2q37 Microdeletion syndrome, 3q29 Microdeletion syndrome, Achondroplasia, ADNP Syndrome, melanocortin 4 receptor (MC4R) disorder, melanocortin 4 receptor (MC4R) deficiency, Proopiomelanocortin Deficiency, 15q24 Microdeletion Syndrome, 15q Duplication Syndrome and Related Disorders, 1p36 Deletion Syndrome, and KANSL1-Related Intellectual Disability Syndrome.

“Improvement in next day functioning” or “wherein there is improvement in next day functioning” refers to improvement after waking from an overnight sleep period wherein the beneficial effect of administration of a vector encoding BDNF applies to at least one symptom of a syndrome herein and is discernable, either subjectively by a patient or objectively by an observer, for a period of time, e.g., 2 hours, 3 hours, 4 hours, 5 hours, 6 hours, 12 hours, 24 hours, etc. after waking.

“PK” refers to the pharmacokinetic profile. Cₘₐₓ is defined as the highest plasma drug concentration estimated during an experiment (ng/ml). Tₘₐₓ is defined as the time when Cₘₐₓ is estimated (min). AUC_{0-∞} is the total area under the plasma drug concentration-time curve, from drug administration until the drug is eliminated (ng·hr/ml or g·hr/ml). The area under the curve is governed by clearance. Clearance is defined as the volume of blood or plasma that is totally cleared of its content of drug per unit time (ml/min).

“Treating”, “treatment” or “treat” can refer to the following: alleviating or delaying the appearance of clinical symptoms of a disease, syndrome or condition in a patient that may be afflicted with or predisposed to the disease, syndrome or condition, but does not yet experience or display clinical or subclinical symptoms of the disease, syndrome or condition. In certain embodiments, “treating”, “treat” or “treatment” may refer to preventing the appearance of clinical symptoms of a disease, syndrome or condition in a patient that may be afflicted with or predisposed to the disease or condition, but does not yet experience or display clinical or subclinical symptoms of the disease, syndrome or condition. “Treating”, “treat” or “treatment” also refers to inhibiting the disease, syndrome or condition, e.g., arresting or reducing its development or at least one clinical or subclinical symptom thereof. “Treating”, “treat” or “treatment” further refers to relieving the disease, syndrome or condition, e.g., causing regression of the disease, syndrome or condition or at least one of its clinical or subclinical symptoms. The benefit to a patient to be treated may be statistically significant, mathematically significant, or at least perceptible to the patient and/or the physician. Nonetheless, prophylactic (preventive) treatment and curative treatment are two separate embodiments of the disclosure herein.

“Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In embodiments, this term refers to molecular entities and compositions approved by a regulatory agency of the federal or a state government, as the GRAS list under section 204(s) and 409 of the Federal Food, Drug and Cosmetic Act, that is subject to premarket review and approval by the FDA or similar lists, the U.S. Pharmacopeia or another generally recognized pharmacopeia for use in animals, and more particularly in humans.

“Effective amount” or “therapeutically effective amount” can mean a dosage sufficient to alleviate one or more symptoms of a syndrome, disorder, disease, or condition being treated, or to otherwise provide a desired pharmacological and/or physiologic effect. “Effective amount” or “therapeutically effective amount” may be used interchangeably herein. In embodiments, the terms “effective amount” or “therapeutically effective amount” refer to an amount of a compound, material, composition, medicament, or other material that is effective to achieve a particular pharmacological and/or physiologic effect in connection with symptoms of a developmental syndrome such as, but not limited to, one or more of the following: reducing or eliminating difficulty in sucking, reducing or eliminating difficulty in feeding, reducing or eliminating poor muscle tone, reducing or eliminating growth hormone deficiency, increasing levels of sex hormones, reducing or eliminating a constant feeling of hunger, reducing or eliminating excessive appetite (hyperphagia), reducing or eliminating weight gain, reducing or eliminating obesity, reducing or eliminating short stature, increasing motor skills, reducing or eliminating occurrence of underdeveloped sex organs, reducing or eliminating intellectual disability, reducing or eliminating learning disability, reducing or eliminating delayed speech development, reducing or eliminating hypothalamic dysphasia, reducing or eliminating delayed language development, reducing or eliminating infertility, reducing or eliminating cognitive rigidity, reducing or eliminating cognitive impairment, reducing or eliminating emotional lability, reducing or eliminating obsessive-compulsive behavior, reducing or eliminating autistic symptomology, reducing or eliminating psychotic episodes, reducing or eliminating bipolar disorder with psychosis, reducing or eliminating excessive daytime sleepiness, reducing or eliminating scoliosis, reducing or eliminating osteopenia/osteoporosis, reducing or eliminating decreased gastrointestinal motility, reducing or eliminating sleep disturbances, and/or reducing or eliminating reduced pain sensitivity, enhancing cognitive function, increasing daytime activity, improving learning (either the rate or ease of learning), improving attention, improving social behavior, and/or improving cerebrovascular function.

In embodiments, effective amount refers to an amount which may be suitable to prevent a decline in any one or more of the above qualities, or, in embodiments, to improve any one or more of the above qualities, for example, hypothalamic dysphasia, constant feeling of hunger, excessive appetite (hyperphagia), weight gain, obesity, cognitive function or performance, learning rate or ability, problem solving ability, attention span and ability to focus on a task or problem, social behavior, and the like. In embodiments, an effective amount may be suitable to reduce either the extent or rate of decline in a subject's appetite dysregulation, weight loss, cognitive skills or functioning, and/or the effective amount may be suitable to delay the onset of such decline. In embodiments, an effective amount may increase hypothalamic BDNF expression. Such effectiveness may be achieved, for example, by administering compositions described herein to an individual or to a population. In embodiments, the reduction, or delay of such a decline, or the improvement in an individual or population can be relative to a cohort, e.g., a control subject or a cohort population that has not received the treatment, or been administered the composition or medicament.

“Co-administered with”, “administered in combination with”, “a combination of” or “administered along with” may be used interchangeably and mean that two or more agents are administered in the course of therapy. The agents may be administered together at the same time or separately in spaced apart intervals. The agents may be administered in a single dosage form or in separate dosage forms.

“Patient in need thereof” may include individuals that have been diagnosed with a developmental syndrome such as Prader-Willi syndrome, 16p11.2 deletion syndrome, 16p11.2 recurrent microdeletion, Albright hereditary osteodystrophy, Alström Syndrome, Bardet-Biedl syndrome, Borjeson-Forssman-Lehmann syndrome, Cohen syndrome, fragile X syndrome, fragile X syndrome (Prader-Willi Subtype), FMR1-Related Disorders, Down syndrome, Klinefelter syndrome, Turner syndrome, Smith-Magenis syndrome, Angelman syndrome, 21-Hydroxylase-Deficient Congenital Adrenal Hyperplasia, 2q37 Microdeletion syndrome, 3q29 Microdeletion syndrome, Achondroplasia, ADNP Syndrome, melanocortin 4 receptor (MC4R) disorder, Proopiomelanocortin Deficiency, 15q24 Microdeletion Syndrome, 15q Duplication Syndrome and Related Disorders, 1p36 Deletion Syndrome, and KANSL1-Related Intellectual Disability Syndrome. In embodiments, the developmental syndrome is melanocortin 4 receptor (MC4R) deficiency. In embodiments, the developmental syndrome manifests hypothalamic dysphasia. The methods may be provided to any individual including, e.g., wherein the patient is a neonate, infant, a pediatric patient (6 months to 12 years), an adolescent patient (age 12-18 years) or an adult (over 18 years). Patients include mammals.

“Prodrug” refers to a pharmacological substance (drug) that is administered to a patient in an inactive (or significantly less active) form. Once administered, the prodrug is metabolized in the body (in vivo) into a compound having the desired pharmacological activity.

“Analog” and “Derivative” may be used interchangeably and refer to a compound that possesses the same core as the parent compound, but may differ from the parent compound in bond order, the absence or presence of one or more atoms and/or groups of atoms, and combinations thereof. Enantiomers are examples of derivatives. The derivative can differ from the parent compound, for example, in one or more substituents present on the core, which may include one or more atoms, functional groups, or substructures. In general, a derivative can be imagined to be formed, at least theoretically, from the parent compound via chemical and/or physical processes.

The term “pharmaceutically acceptable salt”, as used herein, refers to derivatives of the compounds defined herein, wherein the parent compound is modified by making acid or base salts thereof. Example of pharmaceutically acceptable salts include, but are not limited to, nontoxic base addition salts with inorganic bases. Suitable inorganic bases such as alkali and alkaline earth metal bases include metallic cations such as sodium, potassium, magnesium, calcium and the like. The pharmaceutically acceptable salts can be synthesized from the parent compound by conventional chemical methods.

### EXAMPLES

The Examples provided herein are included solely for augmenting the disclosure herein and should not be considered to be limiting in any respect.

### Example I

### Construction of Human BDNF Autoregulatory Vector

Recombinant adeno-associated viral vector construction and packaging. The rAAV plasmid contains a vector expression cassette consisting of the cytomegalovirus enhancer, the chicken ß-actin (CBA) promoter, the woodchuck post-transcriptional regulatory element (WPRE) and bovine growth hormone polyadenosine flanked by AAV inverted terminal repeats. Human BDNF cDNA was fused to the hemagglutinin (HA) tag at the 5′ terminus and then inserted into the multiple cloning sites between the CBA promoter and the WPRE sequence. The genes encoding EGFP or destabilized YFP were cloned into the rAAV plasmid as controls. rAAV serotype 1 vectors were packaged and purified.

microRNA vector construction and adeno-associated vector production. microRNA was used to target BDNF. Two targeting sequences in the BDNF coding region were cloned into the Block-iT PolII miR RNAi expression vector (pcDNA6.2-Gw/miR, Invitrogen). Two targeting sequences with the highest scores (Invitrogene RNAi Design Tool) were selected and cloned into the Block-iT PolII miR RNAi expression vector: WPRE 74: CTATGTGGACGCTGCTTTA [SEQ ID NO:3], and WPRE155: TCCTGGTTTGTCTCTTTAT [SEQ ID NO:4]. In in vitro experiments, both miR constructs inhibited BDNF expression when co-transfected with a BDNF expression plasmid, as confirmed by quantitative PCR and ELISA for BDNF (BDNF Emax ImmunoAssay System, Promega). The miR-Bdnf construct with mature microRNA sequence: 5′-AATACTGTCACACACGCTCAG-3′) [SEQ ID NO:9] was chosen for in vivo experiments. miR-Bdnf and a scrambled microRNA (miR-scr, with the scrambled sequence targeting no known gene, Invitrogen) were subcloned into the rAAV plasmid driven by CBA promoter as described herein.

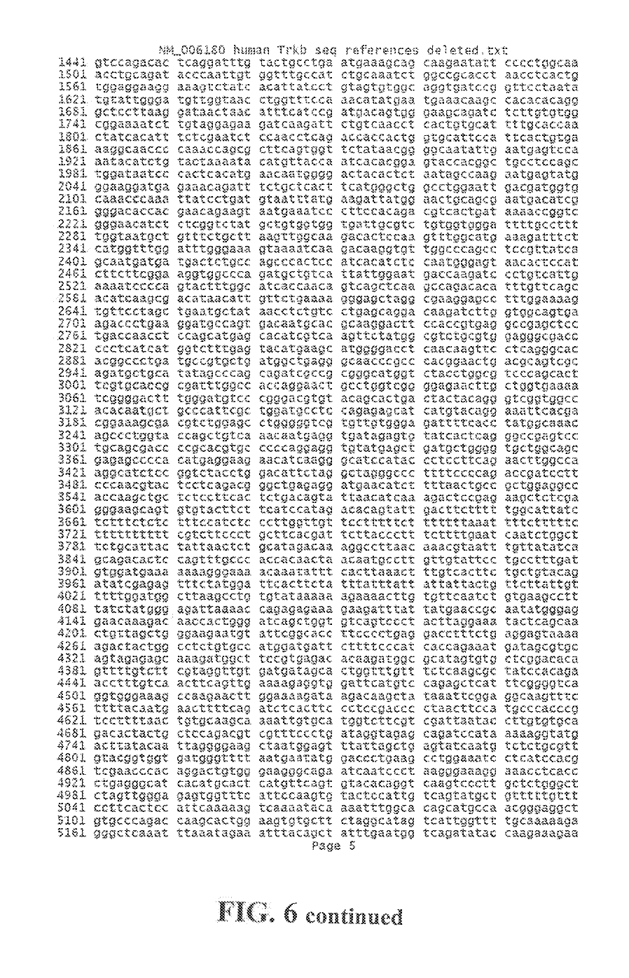

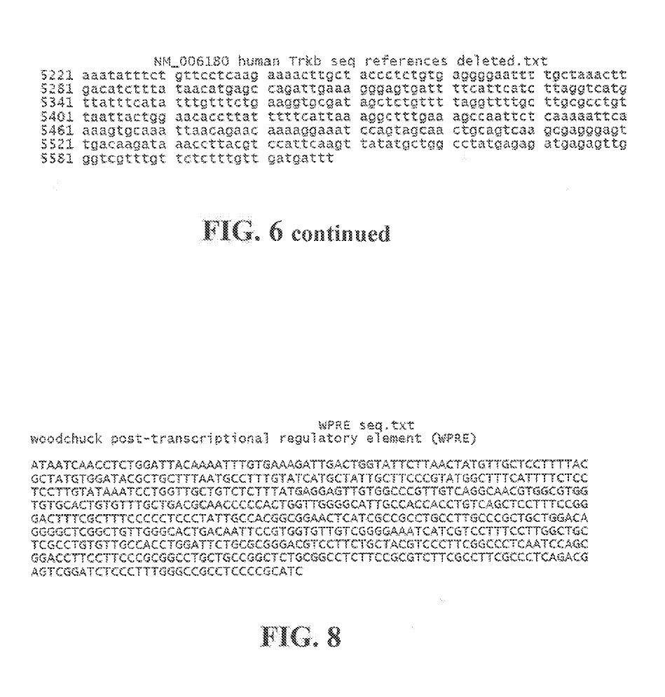

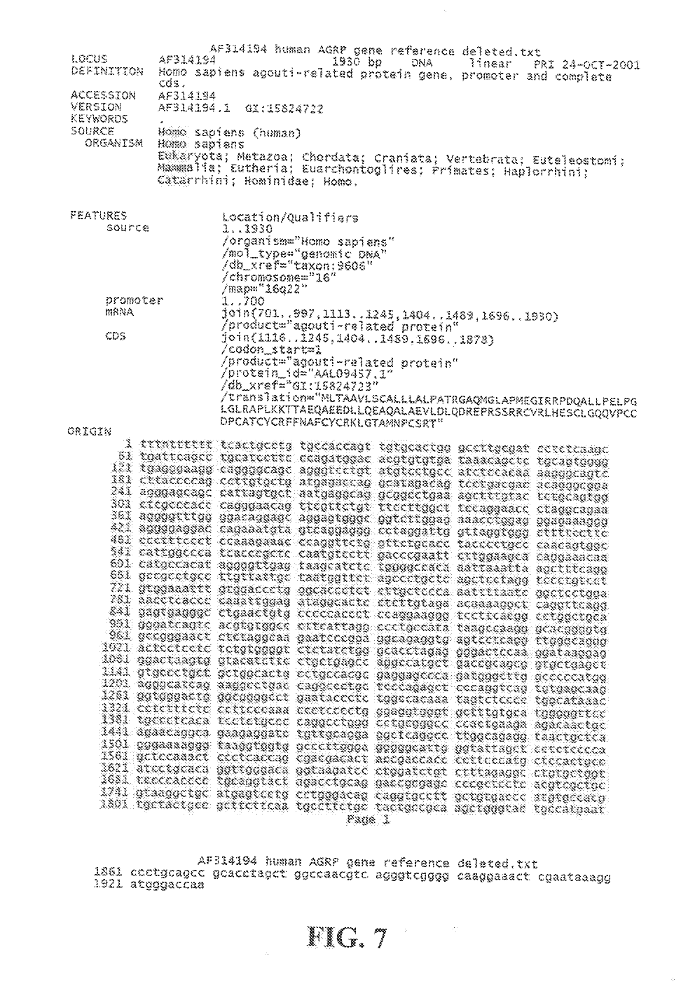

Auto-regulatory system. Two AGRP promoter fragments from human genomic DNA were amplified by PCR. FIG. 7 shows the DNA sequence and gene structure of human AGRP [SEQ ID NO:7]. AGRP484 (484 bp, −133 bp to +351 bp from the start of the noncoding exon28) [see SEQ ID NO:7], and AGRP814 (814 bp, −463/+351) [see SEQ ID NO:7].

### Example 2

### Prospective Assessment of Efficacy of Ultrasound Enhanced Treatment on Melanocortin 4 Receptor (MC4R) Disorder in MC4-R Deficient Mice with Autoregulatory Vectors Encoding BDNF

rAAV autoregulatory plasmids encoding BDNF according to Example 1 (rAAV-BDNF-miR-Bdnf) will be administered to 50 eight week old MC4-R deficient mice (Huszar et al., Cell. 1997 Jan. 10; 88(1):131-41).

Prior to rAAV-BDNF-miR-Bdnf vector administration, ultrasound energy will be applied to the BBB proximate to the hypothalamus. Each mouse will be anesthetized using 2% isoflurane and placed prone with its head immobilized by a stereotaxic apparatus that includes a mouse head holder, ear bars, and a gas anesthesia mask. The mouse hair will be removed using an electric trimmer and a depilatory cream. A degassed water-filled container sealed at the bottom with a thin, acoustically and optically transparent plastic wrap will be placed on top of the mouse head. Ultrasound coupling gel will also be used to eliminate any remaining impedance mismatch.

Ultrasound waves will be generated by a single-element spherical segment focused ultrasound transducer (center frequency: 1.525 MHz, focal depth: 90 mm, radius: 30 mm, available, e.g., from Riverside Research Institute, New York, N.Y., USA). A pulse-echo diagnostic transducer (center frequency: 7.5 MHz, focal length: 60 mm) will be aligned through a central, circular hole (radius 11.2 mm) of the focused ultrasound transducer so that the foci of the two transducers fully overlap. A cone filled with degassed and distilled water will be mounted onto the transducer system with the water contained in the cone by an acoustically transparent polyurethane membrane cap. The transducer system will be attached to a computer-controlled, three-dimensional positioning system (e.g., available from Velmex Inc., Lachine, QC, CAN). The focused ultrasound transducer will be connected to a matching circuit and driven by a computer-controlled function generator and a 50-dB power amplifier. The pulse-echo transducer will be driven by a pulser-receiver system connected to a digitizer in a personal computer.

The focused ultrasound transducer will be submerged in the degassed water-filled container with its beam axis perpendicular to the surface of the skull. The focus of the transducer will be positioned inside the mouse brain using, e.g., a grid-positioning method. The beam axis of the transducer will be aligned such that the focal point is placed 3 mm beneath the top of the parietal bone of the skull. In this placement, the focus of the focused ultrasound beam will overlap with the hypothalamus.

A 25 μl bolus of ultrasound contrast agents constituting of microbubbles (mean diameter: 3.0-4.5 μm, concentration: 5.0-8.0×10⁸ bubbles per ml) will be injected into the tail vein 1-4 minutes prior to sonication. Pulsed focused ultrasound (pulse rate: 10 Hz, pulse duration: 20 ms, duty cycle: 20%) will then be applied at 0.64 MPa peak-to-peak in a series of two bursts consisting of 30 s of sonication at a single location (i.e., the hypothalamus). Between each burst, a 30-s interval will be allowed for any residual heat between pulses to dissipate. The focused ultrasound sonication procedure can be performed one or more times in each mouse brain.

Following BBB opening, a midline incision will be made through the scalp to reveal the skull and two small holes will be drilled into the skull with a dental drill above the injection sites (1.2 mm posterior to the bregma, 0.5 mm lateral to the midline, 6.2 mm dorsal to the bregma). The rAAV vectors (3×10⁹ genomic particles per site) will be injected bilaterally into the hypothalamus at a rate of 0.1 μl min-1 with a 10 μl Hamilton syringe attached to a Micro4 Micro Syringe Pump Controller (World Precision Instruments). At the end of infusion, the syringe will slowly be raised from the brain and the scalp will be sutured.

Primary outcome measures will include the number of mice showing a decrease in fasting total ghrelin from baseline to 1 month following administration of rAAV-BDNF-miR-Bdnf. The following parameters will be measured: body weight, insulin tolerance and glucose tolerance.

The mice will be maintained on a normal 12 h/12 h light/dark cycle with respective diet (normal chow diet (11% fat, 28% protein, 61% carbohydrate, caloric density 3.4 kcal g-l, Research Diets) or high fat diet (45% fat, caloric density 4.73 kcal g-l, Research Diets)) and water ad libitum throughout the experiment. Body weight of each individual mouse will be recorded before injection of rAAV-BDNF-miR-Bdnf and every 3-7 days after injection. Food consumption will be recorded periodically after injection as the total food consumption of each cage housing 4-5 mice and will be represented as the average of food consumption per mouse per day. Blood will be collected from the retroorbital sinus 3-4 weeks after rAAV injection. The mice will be anesthetized at the same time with ketamine (87 mg kg-1)/xylazine (13 mg kg-1) followed by blood withdraw. Serum will be prepared by allowing the blood to clot for 30 min on ice followed by centrifugation. Serum will be at least diluted 1:5 in serum assay diluent and will be assayed using the following DuoSet ELISA Development System (R&D Systems): mouse IGF-1, IGFBP-3, Leptin, Leptin R, Adiponectin/Acrp30. Insulin will be measured using Mercodia ultrasensitive mouse insulin ELISA (ALPCO Diagnostic). Glucose will be measured using QuantiChrom Glucose Assay (BioAssay Systems). Total cholesterol will be measured using Cholesterol E test kit (Wako Diagnostics). Triglyceride will be measured using L-Type test (Wako Diagnostics).

It should be understood that the examples and embodiments provided herein are exemplary examples embodiments. Those skilled in the art will envision various modifications of the examples and embodiments that are consistent with the scope of the disclosure herein. Such modifications are intended to be encompassed by the claims.

1-48. (canceled)

49. A method of treating a developmental syndrome in a patient in need thereof comprising:
applying ultrasound to a target location in the patient's brain to enhance permeability of the patient's blood brain barrier at the target location;
administering to the patient a vector encoding BDNF for delivery of BDNF to the target location, wherein the method provides improvement in at least one symptom of the developmental syndrome.

50. The method according to claim 49, wherein the ultrasound is administered to the target location in the patient's brain at a frequency ranging from 250 kHz to 10 MHz, sonication duration ranging from 100 nanoseconds to 30 minutes, with continuous wave or burst mode operation, where the burst mode repetition varies from 0.01 Hz to 1 MHz.

51. The method according to claim 49, wherein the ultrasound is administered to the target location in the patient's brain at a frequency ranging from 20 kHz to 5 MHz, and with sonication duration ranging from 100 nanoseconds to 1 minute.

52. The method according to claim 49, wherein the ultrasound is administered to the target location in the patient's brain prior to administering the vector.

53. The method according to claim 49, wherein the vector is administered prior to applying the ultrasound.

54. The method according to claim 49, wherein the vector is an adeno-associated virus.

55. The method according to claim 54, wherein the adeno-associated virus is AAV1 or AAV2.

56. The method according to claim 49, wherein the vector is a lentivirus.

57. The method according to claim 49, wherein the vector is administered via a route selected from the group consisting of oral, buccal, sublingual, rectal, topical, intranasal, vaginal and parenteral.

58. The method according to claim 49, wherein the developmental syndrome is selected from the group consisting of Prader-Willi syndrome, 16p11.2 deletion syndrome, 16p11.2 recurrent microdeletion, Albright hereditary osteodystrophy, Alström Syndrome, Bardet-Biedl syndrome, Borjeson-Forssman-Lehmann syndrome, Cohen syndrome, fragile X syndrome, fragile X syndrome (Prader-Willi Subtype), FMR1-Related Disorders, Down syndrome, Klinefelter syndrome, Turner syndrome, Smith-Magenis syndrome, Angelman syndrome, 21-Hydroxylase-Deficient Congenital Adrenal Hyperplasia, 2q37 Microdeletion syndrome, 3q29 Microdeletion syndrome, Achondroplasia, ADNP Syndrome, melanocortin 4 receptor (MC4R) disorder, melanocortin 4 receptor (MC4R) deficiency, Proopiomelanocortin Deficiency, 15q24 Microdeletion Syndrome, 15q Duplication Syndrome and Related Disorders, 1p36 Deletion Syndrome, and KANSL1-Related Intellectual Disability Syndrome.

59. The method according to claim 49, wherein the developmental syndrome is melanocortin 4 receptor (MC4R) disorder.

60. The method according to claim 49, wherein the vector includes (i) a constitutive promoter operatively linked to nucleic acid encoding the BDNF, and (ii) a regulatory sequence including an AGRP promoter operatively linked to an interference RNA sequence, wherein the regulatory sequence down regulates expression of the BDNF in response to BDNF induced physiological changes.

61. The method according to claim 60, wherein the interference RNA sequence is miR-Bdnf (SEQ ID NO:9) operatively linked to WPRE74 (SEQ ID NO. 3) or WPRE155 (SEQ ID NO. 4), the AGRP promoter is AGRP484 or AGRP814, the constitutive promoter is a chicken ß-actin promoter, and the vector further comprises WPRE which is operatively linked to the nucleic acid encoding BDNF.

62. The method according to claim 60, wherein the constitutive promoter is CAG including a CMV early enhancer element, a chicken ß-actin promoter and a splice acceptor of a rabbit beta-globin gene.

63. The method according to claim 60, wherein the vector includes a CMV enhancer and a chicken ß-actin promoter, WPRE and bovine growth hormone poly-A flanked by AAV inverted terminal repeats.

64. The method according to claim 60, wherein the BDNF is flanked by two loxP sites (flox-BDNF), and the BDNF can be knocked out by Cre recombinase.

65. A method of treating a developmental syndrome in a patient in need thereof comprising:
administering to the patient an effective amount of a vector including (i) a constitutive promoter operatively linked to nucleic acid encoding BDNF, and (ii) a regulatory sequence including an AGRP promoter operatively linked to an interference RNA sequence, wherein the regulatory sequence down regulates expression of BDNF in response to BDNF induced physiological changes, and the method provides improvement in at least one symptom of the developmental syndrome.

66. The method of treating a developmental syndrome according to claim 65, wherein the developmental syndrome is selected from the group consisting of Prader-Willi syndrome, 16p11.2 deletion syndrome, 16p11.2 recurrent microdeletion, Albright hereditary osteodystrophy, Alström Syndrome, Bardet-Biedl syndrome, Borjeson-Forssman-Lehmann syndrome, Cohen syndrome, fragile X syndrome, fragile X syndrome (Prader-Willi Subtype), FMR1-Related Disorders, Down syndrome, Klinefelter syndrome, Turner syndrome, Smith-Magenis syndrome, Angelman syndrome, 21-Hydroxylase-Deficient Congenital Adrenal Hyperplasia, 2q37 Microdeletion syndrome, 3q29 Microdeletion syndrome, Achondroplasia, ADNP Syndrome, melanocortin 4 receptor (MC4R) disorder, melanocortin 4 receptor (MC4R) deficiency, Proopiomelanocortin Deficiency, 15q24 Microdeletion Syndrome, 15q Duplication Syndrome and Related Disorders, 1p36 Deletion Syndrome, and KANSL1-Related Intellectual Disability Syndrome.

67. The method according to claim 65, wherein the developmental syndrome is melanocortin 4 receptor (MC4R) disorder.

68. The method of treating a developmental syndrome according to claim 65, wherein the interference RNA sequence is miR-Bdnf (SEQ ID NO:9). operatively linked to WPRE74 (SEQ ID NO. 3) or WPRE155 (SEQ ID NO. 4), the AGRP promoter is AGRP484 or AGRP814, the constitutive promoter is a chicken ß-actin promoter, and the vector further comprises WPRE which is operatively linked to the nucleic acid encoding BDNF.

69. The method of treating a developmental syndrome according to claim 65, wherein the constitutive promoter is CAG including a CMV early enhancer element, a chicken ß-actin promoter and a splice acceptor of a rabbit beta-globin gene.

70. The method of treating a developmental syndrome according to claim 65, wherein the vector includes a CMV enhancer and a chicken ß-actin promoter, WPRE and bovine growth hormone poly-A flanked by AAV inverted terminal repeats.

71. The method of treating a developmental syndrome according to claim 65, wherein the BDNF is flanked by two loxP sites (flox-BDNF), and the BDNF can be knocked out by Cre recombinase.

72. The method of treating a developmental syndrome according to claim 65, wherein the vector is administered via a route selected from the group consisting of oral, buccal, sublingual, rectal, topical, intranasal, vaginal and parenteral.

73. The method of treating a developmental syndrome according to claim 65, further comprising administering ultrasound to a target location in the patient's brain to enhance permeability of the patient's blood brain barrier at the target location.


SEQUENCE LISTING (12 sequences)

SEQ ID NO: 1 - DNA, 5641 bp, Artificial Sequence
  Description of Artificial Sequence Synthetic polynucleotide
tagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtaccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccgccatgctaggtaacaagcgactggggctgtccggactgaccctcgccctgtccctgctcgtgtgcctgggtgcgctggccgaggcgtacccctccaagccggacaacccgggcgaggacgcaccagcggaggacatggccagatactactcagcgctgcgacactacatcaacctcatcaccaggcagagatatggaaaacgatctagcccagagacactgatttcagacctcttgatgagagaaagcacagaaaatgttcccagaactcggcttgaagaccctgcaatgtggtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattc

SEQ ID NO: 2 - DNA, 2365 bp, Artificial Sequence
  Description of Artificial Sequence Synthetic polynucleotide
cattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccactcgagtggagctcgcgactagtcgattcgaattcggcttgtgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatatccgtatgatgttcctgattattgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctcccc

SEQ ID NO: 3 - DNA, 19 bp, Artificial Sequence
  Description of Artificial Sequence Synthetic oligonucleotide
ctatgtggacgctgcttta

SEQ ID NO: 4 - DNA, 19 bp, Artificial Sequence
  Description of Artificial Sequence Synthetic oligonucleotide
tcctggtttgtctctttat

SEQ ID NO: 5 - DNA, 4755 bp, Homo sapiens
cacacacacacacacacacagagagaacatctctagtaaaaagaaaagttgagctttcttagctagatgtgtgtattagccagaaaaagccaaggagtgaagggttttagagaactggaggagataaagtggagtctgcatatgggaggcatttgaaatggacttaaatgtctttttaatgctgactttttcagttttctccttaccagacacattgttttcatgacattagccccaggcatagacacatcattaaaatgaacatgtcaaaaaatgatttctgtttagaaataagcaaaacattttcagttgtgaccacccaggtgtagaataaagaacagtggaattgggagccctgagttctaacataaactttcttcatgacataaggcaagtcttctatggcctttggtttccttacctgtaaaacaggatggctcaatgaaattatctttcttctttgctataatagagtatctctgtgggaagaggaaaaaaaaagtcaatttaaaggctccttatagttccccaactgctgttttattgtgctattcatgcctagacatcacatagctagaaaggcccatcagacccctcaggccactgctgttcctgtcacacattcctgcaaaggaccatgttgctaacttgaaaaaaattactattaattacacttgcagttgttgcttagtaacatttatgattttgtgtttctcgtgacagcatgagcagagatcattaaaaattaaacttacaaagctgctaaagtgggaagaaggagaacttgaagccacaatttttgcacttgcttagaagccatctaatctcaggttatatgctagatcttgggggcaaacactgcatgtctctggtttatattaaaccacatacagcacactactgacactgatttgtgtctggtgcagctggagtttatcaccaagacataaaaaaaccttgaccctgcagaatggcctggaattacaatcagatgggccacatggcatcccggtgaaagaaagccctaaccagttttctgtcttgtttctgctttctccctacagttccaccaggtgagaagagtgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagtggatttatgttgtatagattagattatattgagacaaaaattatctatttgtatatatacataacagggtaaattattcagttaagaaaaaaataattttatgaactgcatgtataaatgaagtttatacagtacagtggttctacaatctatttattggacatgtccatgaccagaagggaaacagtcatttgcgcacaacttaaaaagtctgcattacattccttgataatgttgtggtttgttgccgttgccaagaactgaaaacataaaaagttaaaaaaaataataaattgcatgctgctttaattgtgaattgataataaactgtcctctttcagaaaacagaaaaaaacacacacacacacaacaaaaatttgaaccaaaacattccgtttacattttagacagtaagtatcttcgttcttgttagtactatatctgttttactgcttttaacttctgatagcgttggaattaaaacaatgtcaaggtgctgttgtcattgctttactggcttaggggatgggggatggggggtatatttttgtttgttttgtgtttttttttcgtttgtttgttttgttttttagttcccacagggagtagagatggggaaagaattcctacaatatatattctggctgataaaagatacatttgtatgttgtgaagatgtttgcaatatcgatcagatgactagaaagtgaataaaaattaaggcaactgaacaaaaaaatgctcacactccacatcccgtgatgcacctcccaggccccgctcattctttgggcgttggtcagagtaagctgcttttgacggaaggacctatgtttgctcagaacacattctttccccccctccccctctggtctcctctttgttttgttttaaggaagaaaaatcagttgcgcgttctgaaatattttaccactgctgtgaacaagtgaacacattgtgtcacatcatgacactcgtataagcatggagaacagtgatttttttttagaacagaaaacaacaaaaaataaccccaaaatgaagattattttttatgaggagtgaacatttgggtaaatcatggctaagcttaaaaaaaactcatggtgaggcttaacaatgtcttgtaagcaaaaggtagagccctgtatcaacccagaaacacctagatcagaacaggaatccacattgccagtgacatgagactgaacagccaaatggaggctatgtggagttggcattgcatttaccggcagtgcgggaggaatttctgagtggccatcccaaggtctaggtggaggtggggcatggtatttgagacattccaaaacgaaggcctctgaaggacccttcagaggtggctctggaatgacatgtgtcaagctgcttggacctcgtgctttaagtgcctacattatctaactgtgctcaagaggttctcgactggaggaccacactcaagccgacttatgcccaccatcccacctctggataattttgcataaaattggattagcctggagcaggttgggagccaaatgtggcatttgtgatcatgagattgatgcaatgagatagaagatgtttgctacctgaacacttattgctttgaaactagacttgaggaaaccagggtttatcttttgagaacttttggtaagggaaaagggaacaggaaaagaaaccccaaactcaggccgaatgatcaaggggacccataggaaatcttgtccagagacaagacttcgggaaggtgtctggacattcagaacaccaagacttgaaggtgccttgctcaatggaagaggccaggacagagctgacaaaattttgctccccagtgaaggccacagcaaccttctgcccatcctgtctgttcatggagagggtccctgcctcacctctgccattttgggttaggagaagtcaagttgggagcctgaaatagtggttcttggaaaaatggatccccagtgaaaactagagctctaagcccattcagcccatttcacacctgaaaatgttagtgatcaccacttggaccagcatccttaagtatcagaaagccccaagcaattgctgcatcttagtagggtgagggataagcaaaagaggatgttcaccataacccaggaatgaagataccatcagcaaagaatttcaatttgttcagtctttcatttagagctagtctttcacagtaccatctgaatacctctttgaaagaaggaagactttacgtagtgtagatttgttttgtgttgtttgaaaatattatctttgtaattatttttaatatgtaaggaatgcttggaatatctgctatatgtcaactttatgcagcttccttttgagggacaaatttaaaacaaacaaccccccatcacaaacttaaaggattgcaagggccagatctgttaagtggtttcataggagacacatccagcaattgtgtggtcagtggctcttttacccaataagatacatcacagtcacatgcttgatggtttatgttgacctaagatttattttgttaaaatctctctctgttgtgttcgttcttgttctgttttgttttgttttttaaagtcttgctgtggtctctttgtggcagaagtgtttcatgcatggcagcaggcctgttgcttttttatggcgattcccattgaaaatgtaagtaaatgtctgtggccttgttctctctatggtaaagatattattcaccatgtaaaacaaaaaacaatatttattgtattttagtatatttatataattatgttattgaaaaaaattggcattaaaacttaaccgcatcagaacctattgtaaatacaagttctatttaagtgtactaattaacatataatatatgttttaaatatagaatttttaatgtttttaaatatattttcaaagtacataaaa

SEQ ID NO: 6 - DNA, 5608 bp, Homo sapiens
aagacggattctcagacaaggcttgcaaatgccccgcagccatcatttaactgcacccgcagaatagttacggtttgtcacccgaccctcccggatcgcctaatttgtccctagtgagaccccgaggctctgcccgcgcctggcttcttcgtagctggatgcatatcgtgctccgggcagcgcgggcgcagggcacgcgttcgcgcacaccctagcacacatgaacacgcgcaagagctgaaccaagcacggtttccatttcaaaaagggagacagcctctaccgcgattgtagaagagactgtggtgtgaattagggaccgggaggcgtcgaacggaggaacggttcatcttagagactaattttctggagtttctgcccctgctctgcgtcagccctcacgtcacttcgccagcagtagcagaggcggcggcggcggctcccggaattgggttggagcaggagcctcgctggctgcttcgctcgcgctctacgcgctcagtccccggcggtagcaggagcctggacccaggcgccgccggcgggcgtgaggcgccggagcccggcctcgaggtgcataccggacccccattcgcatctaacaaggaatctgcgccccagagagtcccgggagcgccgccggtcggtgcccggcgcgccgggccatgcagcgacggccgccgcggagctccgagcagcggtagcgcccccctgtaaagcggttcgctatgccggggccactgtgaaccctgccgcctgccggaacactcttcgctccggaccagctcagcctctgataagctggactcggcacgcccgcaacaagcaccgaggagttaagagagccgcaagcgcagggaaggcctccccgcacgggtgggggaaagcggccggtgcagcgcggggacaggcactcgggctggcactggctgctagggatgtcgtcctggataaggtggcatggacccgccatggcgcggctctggggcttctgctggctggttgtgggcttctggagggccgctttcgcctgtcccacgtcctgcaaatgcagtgcctctcggatctggtgcagcgacccttctcctggcatcgtggcatttccgagattggagcctaacagtgtagatcctgagaacatcaccgaaattttcatcgcaaaccagaaaaggttagaaatcatcaacgaagatgatgttgaagcttatgtgggactgagaaatctgacaattgtggattctggattaaaatttgtggctcataaagcatttctgaaaaacagcaacctgcagcacatcaattttacccgaaacaaactgacgagtttgtctaggaaacatttccgtcaccttgacttgtctgaactgatcctggtgggcaatccatttacatgctcctgtgacattatgtggatcaagactctccaagaggctaaatccagtccagacactcaggatttgtactgcctgaatgaaagcagcaagaatattcccctggcaaacctgcagatacccaattgtggtttgccatctgcaaatctggccgcacctaacctcactgtggaggaaggaaagtctatcacattatcctgtagtgtggcaggtgatccggttcctaatatgtattgggatgttggtaacctggtttccaaacatatgaatgaaacaagccacacacagggctccttaaggataactaacatttcatccgatgacagtgggaagcagatctcttgtgtggcggaaaatcttgtaggagaagatcaagattctgtcaacctcactgtgcattttgcaccaactatcacatttctcgaatctccaacctcagaccaccactggtgcattccattcactgtgaaaggcaaccccaaaccagcgcttcagtggttctataacggggcaatattgaatgagtccaaatacatctgtactaaaatacatgttaccaatcacacggagtaccacggctgcctccagctggataatcccactcacatgaacaatggggactacactctaatagccaagaatgagtatgggaaggatgagaaacagatttctgctcacttcatgggctggcctggaattgacgatggtgcaaacccaaattatcctgatgtaatttatgaagattatggaactgcagcgaatgacatcggggacaccacgaacagaagtaatgaaatcccttccacagacgtcactgataaaaccggtcgggaacatctctcggtctatgctgtggtggtgattgcgtctgtggtgggattttgccttttggtaatgctgtttctgcttaagttggcaagacactccaagtttggcatgaaagatttctcatggtttggatttgggaaagtaaaatcaagacaaggtgttggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccagtactttggcatcaccaacagtcagctcaagccagacacatttgttcagcacatcaagcgacataacattgttctgaaaagggagctaggcgaaggagcctttggaaaagtgttcctagctgaatgctataacctctgtcctgagcaggacaagatcttggtggcagtgaagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtctacctggacattctaggctagggcccttttccccagaccgatccttcccaacgtactcctcagacgggctgagaggatgaacatcttttaactgccgctggaggccaccaagctgctctccttcactctgacagtattaacatcaaagactccgagaagctctcgagggaagcagtgtgtacttcttcatccatagacacagtattgacttctttttggcattatctctttctctctttccatctcccttggttgttcctttttctttttttaaattttctttttcttttttttttcgtcttccctgcttcacgattcttaccctttcttttgaatcaatctggcttctgcattactattaactctgcatagacaaaggccttaacaaacgtaatttgttatatcagcagacactccagtttgcccaccacaactaacaatgccttgttgtattcctgcctttgatgtggatgaaaaaaagggaaaacaaatatttcacttaaactttgtcacttctgctgtacagatatcgagagtttctatggattcacttctatttatttattattattactgttcttattgtttttggatggcttaagcctgtgtataaaaaagaaaacttgtgttcaatctgtgaagcctttatctatgggagattaaaaccagagagaaagaagatttattatgaaccgcaatatgggaggaacaaagacaaccactgggatcagctggtgtcagtccctacttaggaaatactcagcaactgttagctgggaagaatgtattcggcaccttcccctgaggacctttctgaggagtaaaaagactactggcctctgtgccatggatgattcttttcccatcaccagaaatgatagcgtgcagtagagagcaaagatggcttccgtgagacacaagatggcgcatagtgtgctcggacacagttttgtcttcgtaggttgtgatgatagcactggtttgtttctcaagcgctatccacagaacctttgtcaacttcagttgaaaagaggtggattcatgtccagagctcatttcggggtcaggtgggaaagccaagaacttggaaaagataagacaagctataaattcggaggcaagtttcttttacaatgaacttttcagatctcacttccctccgacccctaacttccatgcccacccgtccttttaactgtgcaagcaaaattgtgcatggtcttcgtcgattaataccttgtgtgcagacactactgctccagacgtcgtttccctgataggtagagcagatccataaaaaggtatgacttatacaattaggggaagctaatggagtttattagctgagtatcaatgtctctgcgttgtacggtggtgatgggttttaatgaatatggaccctgaagcctggaaatcctcatccacgtcgaacccacaggactgtgggaagggcagaatcaatccctaagggaaaggaaacctcaccctgagggcatcacatgcactcatgttcagtgtacacaggtcaagtcccttgctctgggctctagttgggagagtggtttcattccaagtgtactccattgtcagtatgctgtttttgtttccttcactccattcaaaaagtcaaaatacaaaatttggcacagcatgccaacgggaggctgtgcccagaccaagcactggaagtgtgcttctaggcatagtcattggttttgcaaaaagagggctcaaatttaaatagaaatttacagctatttgaatggtcagatataccaagaaagaaaaatatttctgttcctcaagaaaacttgctaccctctgtgaggggaattttgctaaacttgacatctttataacatgagccagattgaaagggagtgattttcattcatcttaggtcatgttatttcatatttgtttctgaaggtgcgatagctctgttttaggttttgcttgcgcctgttaattactggaacaccttatttttcattaaaggctttgaaagccaattctcaaaaattcaaaagtgcaaattaacagaacaaaaggaaatccagtagcaactgcagtcaagcgagggagttgacaagataaaccttacgtccattcaagttatatgctggcctatgagagatgagagttgggtcgtttgttctctttgttgatgattt

SEQ ID NO: 7 - DNA, 1930 bp, Homo sapiens
  modified_base (4)..(4): a, c, g, t, unknown or other
  misc_feature (4)..(4): n is a, c, g, or t
tttntttttttcactgcctgtgccaccagttgtgcactgggccttgcgatcctctcaagctgattcagcctgcatccttcccagatggacacgtgtgtgataaacagctctgcagtggggtgagggaaggcaggggcagcagggtcctgtatgtcctgccatctccacaaaagggcagtccttaccccagccttgtgctgatgagaccaggcatagacagtcctgacgacacagggcggaagggagcagccattagtgctaatgaggcaggcggcctgaaagctttgtactctgcagtggctcgcccacccagggaacagttcgttctgtttccttggcttccaggaaccctaggcagaaaggggtttggggacaggagcaggagtgggcggtcttggagaaacctggagggagaaagggaggggaggaccagaaatgtagtcaggagggcctaggattggttaggtgggcttttccttcccctttccctccaaagaaacccaggttctggttctgcacctacccctgcccaacagtggccattggcccatcacccgctccaatgtccttgacccgaattcttggaagcacaggaaacaacatgccacataggggttgagtaagcatctctggggccacaaattaaattaagctttcagggccgcctgccttgttattgctaatggttctagccctgctcagctcctaggtccctgtcctgtggaaatttgtggaccctgggcaccctctcttgctcccaaattttaatcggctcctggaaacctcaccccaaattggagataggcactcctcttgtagaacaaaaggctcaggttcagggagtgagggcctgaactgtgcccccaccctccaggaagggtccttcacggcctggctgcagggatcagtcacgtgtggcccttcattaggccctgccatataagccaagggcacggggtggccgggaactctctaggcaagaatcccggaggcagaggtgagtcctcaggttgggcagggactcctcctctctgtggggtctctatctgggcacctagaggggactccaaggataaggagggactaagtggtacatcttcctgctgagccaggccatgctgaccgcagcggtgctgagctgtgccctgctgctggcactgcctgccacgcgaggagcccagatgggcttggcccccatggagggcatcagaaggcctgaccaggccctgctcccagagctcccaggtcagtgtgagcaagggtgggactgggcggggcctgaataccctctggccacaaatagtctcccctggcataaaccctctttctcccttcccaaaccctcccctgggaggtgggtgctttgtgcatgggggttcctgccctcacatcctctgccccaggcctgggcctgcgggccccactgaagaagacaactgcagaacaggcagaagaggatctgttgcaggaggctcaggccttggcagaggtaactgctcagggaaaagggtaaggtggtggcccttgggagggggcattgggtattagctcctctccccagctccaaactccctcaccagcgacgacactaccgaccaccccttcccatgctccactgccatcctgcacaggttgggacaggtaagatccctggatctgtctttagaggcctgtgctggttccccacccctgcaggtactagacctgcaggaccgcgagccccgctcctcacgtcgctgcgtaaggctgcatgagtcctgcctgggacagcaggtgccttgctgtgacccatgtgccacgtgctactgccgcttcttcaatgccttctgctactgccgcaagctgggtactgccatgaatccctgcagccgcacctagctggccaacgtcagggtcggggcaaggaaactcgaataaaggatgggaccaa

SEQ ID NO: 8 - DNA, 594 bp, Unknown
  Description of Unknown Woodchuck post- transcriptional regulatory element polynucleotide
ataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatc

SEQ ID NO: 9 - DNA, 21 bp, Artificial Sequence
  Description of Artificial Sequence Synthetic oligonucleotide
aatactgtcacacacgctcag

SEQ ID NO: 10 - protein, 247 aa, Homo sapiens
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 11 - protein, 838 aa, Homo sapiens
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKDFSWFGFGKVKSRQGVGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 12 - protein, 132 aa, Homo sapiens
MLTAAVLSCALLLALPATRGAQMGLAPMEGIRRPDQALLPELPGLGLRAPLKKTTAEQAEEDLLQEAQALAEVLDLQDREPRSSRRCVRLHESCLGQQVPCCDPCATCYCRFFNAFCYCRKLGTAMNPCSRT